(19) United States
(12) Patent Application Publication
(10) Pub. No.: US 2026/0124225 A1
(43) Pub. Date: May 7, 2026

(54) THERAPEUTIC COMPOSITIONS AND METHODS FOR USING SAME

(71) Applicant: William Karnick, Newport Beach, CA (US)

(72) Inventor: William Karnick, Newport Beach, CA (US)

(21) Appl. No.: 19/380,814

(22) Filed: Nov. 5, 2025

Related U.S. Application Data
(60) Provisional application No. 63/717,206, filed on Nov. 6, 2024.

(51) Int. Cl. A61K 31/7048; A61K 31/352

(52) U.S. Cl. CPC A61K 31/7048; A61K 31/352

(57) ABSTRACT

The present invention provides compositions that can include naturally derived compounds such as myricetin (or a derivative or salt thereof) or an anthocyanin (or a derivative or salt thereof) or a combination of thereof. The compositions can be used in a variety of applications such as for managing weight, treating a metabolic disorder, maintaining cardiovascular health, reducing systemic inflammation, and/or treating addiction in a subject.

### CROSS-REFERENCES TO RELATED APPLICATIONS

This application claims priority to and benefit of U.S. Provisional Patent Application 63/717,206, entitled “Therapeutic Compositions and Methods for Using Same,” and filed Nov. 6, 2024, the contents of which are incorporated by reference in their entirety for all purposes.

### FIELD OF THE INVENTION

The present invention relates to the fields of medicine, health, and/or nutrition. More particularly, the present invention relates to a composition that can include one or more high purity, naturally derived compounds such as myricetin (or a derivative or salt thereof) and/or an anthocyanin (or a derivative or salt thereof) (e.g., cyanidin 3-glucoside (C3G)) or a combination thereof. The composition can be used in a variety of applications such as for managing weight, treating a metabolic disorder, maintaining cardiovascular health, reducing systemic inflammation, and/or treating addiction and/or alcoholism in a subject.

### BACKGROUND OF THE INVENTION

Many treatment protocols involve administration of synthetic drugs by injection which can be expensive and have numerous side effects. For example, in the field of weight management, most current glucagon-like peptide-1 (GLP-1) agonist drugs are synthetic injectables with uncomfortable side effects that require a medical visit, diagnosis of diabetes or obesity, and/or a doctor's prescription. Most people prefer oral administration to self-injection. In a 2022 survey of 2,098 participants, 63.2% reported experiencing needle phobia and 94.1% of those reporting needle phobia prefer using non-invasive alternatives (Alsbrooks, K., & Hoerauf, K. (2022). Prevalence, causes, impacts, and management of needle phobia: An international survey of a general adult population. PloS one, 17 (11), e0276814). In addition, most current users experience significant muscle loss as a percentage of overall weight loss, which can be problematic to the overall health of the person. By way of example, muscle loss can lead to muscle weakness, which can result in difficulty of performing daily activities such as walking, climbing stairs, etc. Muscle loss can also lead to increased falls to a person, which can lead to injuries such as bone fractures.

So while current GLP-1 agonist drugs can help with weight management, problems associated with their source (e.g., synthetic), delivery (e.g., injection), side effects, and/or muscle loss or muscle atrophy can be detrimental to the person.

### BRIEF SUMMARY OF THE INVENTION

A solution to at least one or more of the aforementioned problems associated with synthetic injectable GLP-1 agonist drugs has been discovered. In one aspect, the solution can include a combination of naturally derived compounds such as myricetin (or a derivative or salt thereof) and an anthocyanin (or a derivative or salt thereof) that can be used to reduce the overall weight of a subject (e.g., human or animal) while preserving and/or limiting muscle loss of the subject. In one particular aspect, myricetin and the anthocyanin cyanidin 3-glucoside (C3G) can be effective in reducing a person's weight and/or limiting muscle loss. A weight ratio of myricetin and the anthocyanin (e.g., C3G) of 10:1 to 1:10, or preferably 8:1 to 3:1, or more preferably about 6.25 or 5:1 can be particularly effective. This combination of myricetin and an anthocyanin (e.g., C3G) provides a technical advantage over current GLP-1 agonists. For instance, whereas most current GLP-1 agonist treatments are synthetic injectables, can have significant side effects, and result in substantial muscle loss in the subject, the compositions of the present invention relate to a naturally derived, orally administered GLP-1 agonist composition with lesser side effects that also mitigates and/or protects against muscle loss in the subject. Still further, this combination of ingredients helps with patient compliance, particularly with those subjects who cannot tolerate the gastrointestinal distress caused by existing solutions, and those subjects who may have anxiety towards other forms of administration (e.g., injection). In another aspect, the combination of myricetin and an anthocyanin or derivatives or salt forms thereof (e.g., C3G) can be used to help treat metabolic and/or diabetic disorders, maintain cardiovascular health, reduce systemic inflammation, and/or treat addiction and/or alcoholism in a subject.

Disclosed herein, in some aspects, is a composition that can include therapeutically effective amounts of myricetin or a derivative or salt thereof and an anthocyanin or a derivative or salt thereof. In some aspects, the anthocyanin is C3G or cyanidin 3-O glucoside.

In some aspects, the composition is capable of activating a MAPK pathway, a PI3K/AKT pathway, a JAK/STAT pathway, a TGF-β pathway, an AMPK pathway, a Wnt/β-catenin pathway, and/or a melaocortin signaling pathway. In some aspects, the composition is capable of inhibiting dipeptidyl peptidase 4 (DPPV-4). In some aspects, the composition is capable of causing insulin secretion, glucose regulation, appetite regulation, adropin elevation, energy homeostasis, mitochondrial function enhancement, thermogenesis, Sirt3 activation, upregulation of UCP1, mitochondrial biogenesis, lipid metabolism modulation, activation of PPARalpha, fatty acid oxidation, suppression of lipogenesis (e.g., via SREBP-1c downregulation), adipose tissue browning, increased energy expenditure, induction of beige adipocytes, activation of β3-adrenergic receptors, anti-inflammatory effects, reduction of oxidative stress, reduction of proinflammatory cytokines, increase in anti-inflammatory adipokines, mitigation of oxidative stress, modulation of neuroendocrine pathways, influence on hypothalamic AMPK pathways, interaction with opioid receptors, and/or the like, or any combination thereof.

In some aspects, the composition is capable of activating a GLP-1 receptor and/or any receptor associated with metabolism, metabolic diseases and/or addiction. The term “associated with” in the context of receptors refers to a receptor that is known to have an effect on a characteristic or condition or disease, etc. when activated or inhibited. For example, if a receptor is associated with a disease, its activation or inhibition can influence the development of the disease.

In some aspects, the composition includes about 100 mg to about 5000 mg myricetin or derivative or salt thereof and about 50 mg to about 3000 mg anthocyanin or derivative or salt thereof, or any derivable range therein, such as, for example, about 1875 or 1500 mg myricetin or the derivative or salt thereof and about 300 mg anthocyanin or the derivative or salt thereof.

In some aspects, the ratio of myricetin or the derivative or salt thereof to an anthocyanin or the derivative or salt thereof is about 1:10 to about 20:1 by weight, or any derivable range therein. In some aspects, the ratio of myricetin or the derivative or salt thereof to an anthocyanin or the derivative or salt thereof is about 3:1 to 7:1, 4:1 to 6:1, 4.5:1 to 5.5:1, or about 5:1 by weight. In some aspects, the ratio of myricetin or the derivative or salt thereof to an anthocyanin or the derivative or salt thereof is about 8.25:1 to 4.25:1, 8:1 to 4.5:1, 7.75:1 to 4.75:1, 7.5:1 to 5:1, 7.25:1 to 5.25:1, 7:1 to 5.5:1, 6.75:1 to 5.75:1, 6.5:1 to 6:1, or about 6.25:1 by weight.

In some aspects, the composition is encapsulated. In some aspects, the composition is in modified-release form.

In some aspects, the composition is in oral dosage form. In some aspects, the oral dosage form is a capsule, a tablet, a caplet, a gel caplet (gelcap), a syrup, a liquid, or a powder, or the like, or any combination thereof. In some aspects, the oral dosage form is a chewable form, a swallowable form, a dissolvable form, an effervescent, a granulated form, an oral liquid solution, or the like, or any combination thereof.

In some aspects, the composition is any of the compositions disclosed herein in a form modified for increased bioavailability. The disclosure includes any equivalent formulation that has been modified to increase bioavailability and therefore may be, for example, provided at a lower dosage.

In some aspects, the composition is for weight management, treatment of a metabolic disorder, and/or treatment of an addiction in a subject. In some aspects, the weight management includes one or more of weight loss, maintenance of weight loss, preserving lean body mass, minimizing loss of lean body mass during weight loss, decreased food consumption, increasing satiety, reducing pre-meal hunger, reducing intra-meal food intake, prevention of weight gain in the subject, or the like, or any combination thereof. In some aspects, the metabolic disorder is selected from is diabetes, a diabetes-related disorder, obesity, or an obesity-related disorder. In some aspects, the addiction is an opioid addiction or an alcohol addiction or the like or any combination thereof. In some aspects, the subject is a juvenile (e.g., 17 years old or less) or an adult (e.g., 18 years old or greater). In some aspects, the subject is obese, overweight, or suspected to gain weight.

In some aspects, the myricetin or a derivative or salt thereof or the anthocyanin and/or a derivative or salt thereof is at least 80%, 85%, 90%, 95%, 96%, 97%, 98%, 99%, 99.5%, or more pure. By way of example, when the myricetin or a derivative or salt thereof and/or the anthocyanin or a derivative or salt thereof is at least 80% pure, the myricetin or a derivative or salt thereof compound and/or the anthocyanin or a derivative or salt thereof compound is at least 80% by weight of the myricetin or a derivative or salt thereof and/or the anthocyanin or a derivative or salt thereof.

In some aspects, the myricetin or the derivative or salt thereof and the anthocyanin or the derivative or salt thereof act synergistically. The term “synergistically” refers to the effect of two or more compounds when used in combination, which is higher than the sum of the effects when using two or more compounds alone.

In some aspects, the composition does not include quercetin, and/or chlorogenic acid.

Disclosed herein, in some aspects, is a dietary supplement comprising one of any of the compositions disclosed herein in a consumable carrier. In some aspects, the dietary supplement is at least 75% natural and/or plant-based. In some aspects, the dietary supplement is 100% natural and/or plant-based.

Disclosed herein, in some aspects, is a pharmaceutical composition comprising one of any of the compositions disclosed herein in a pharmaceutically acceptable carrier.

Disclosed herein, in some aspects, is a food additive comprising one of any of the compositions disclosed herein.

Disclosed herein, in some aspects, is a food product comprising one of any of the compositions disclosed herein comprising a consumable carrier. In some aspects, the consumable carrier is a cookie, a brownie, a cracker, a breakfast bar, an energy bar, cereal, cake, beverage or the like, or any combination thereof.

In some aspects, the dietary supplement, food additive, or the food product further includes a natural dietary fiber. In some aspects, the dietary fiber contributes to the bioavailability of the composition. In some aspects, the dietary supplement is indigestible dextrin, polydextrose, soybean-derived water-soluble dietary fiber, hydrolyzed guar gum, glucomannan, inulin, pectin, or sodium alginate, or the like, or any combination thereof.

Disclosed herein, in some aspects, is a method for managing weight and/or treating a metabolic disorder and/or addiction in a subject. In some aspects, the method includes administering any one of the compositions, dietary supplements, pharmaceutical compositions, food additives, and/or the food products disclosed herein to the subject.

Disclosed herein, in some aspects, is a method for activating a glucagon-like peptide-1 receptor in a subject. In some aspects, the method includes administering any one of the compositions disclosed herein to the subject, wherein the glucagon-like peptide-1 receptor in the subject is activated. In some aspects, the insulin production in the subject is increased after administration of the composition to the subject.

Disclosed herein, in some aspects, is a method of reducing weight or preventing/minimizing weight gain of a subject. In some aspects, the method includes administering the any one of the compositions disclosed herein to the subject, wherein the subject's weight is reduced or weight gain in a subject is prevented/minimized. In some aspects, the subject' lean muscle mass is preserved.

In some aspects, the managing weight comprises one or more of weight loss, maintenance of weight loss, preserving lean body mass, minimizing loss of lean body mass during weight loss, decreased food consumption, increasing satiety, reducing pre-meal hunger, reducing intra-meal food intake, preventing weight gain, or minimizing weight gain.

In some aspects, the metabolic disorder is diabetes, a diabetes-related disorder, obesity, or an obesity-related disorder. In some aspects, the, (i) the diabetes related disorder is insulin resistance, glucose intolerance, increased fasting glucose, pre-diabetes, type 1 diabetes, type 2 diabetes, gestational diabetes, hypertension, dyslipidemia, fatty liver disease or the like or any combination thereof, (ii) the diabetes-related disorder is atherosclerosis, arteriosclerosis, coronary heart disease, peripheral artery disease and stroke; or is associated with atherogenic dyslipidemia, blood fat disorders, elevated blood pressure, hypertension, a prothrombotic state, a proinflammatory state, or the like, or a combination thereof, or (iii) the obesity related disorder is selected from obesity linked inflammation, obesity linked gallbladder disease, obesity induced sleep apnea, or the like, or any combination thereof.

In some aspects, the addiction is alcoholism or addiction to opioids.

In some aspects of the method, the anthocyanin is cyanidin 3 glucoside or cyanidin 3-O glucoside.

In some aspects of the method, the composition comprises about 100 mg to about 5000 mg myricetin or the derivative or salt thereof and about 50 mg to about 3000 mg anthocyanin or the derivative or salt thereof, or any derivable range therein. In some aspects, the composition comprises about 1500 mg myricetin or the derivative or salt thereof and about 300 mg anthocyanin or the derivative or salt thereof. In some aspects, the composition comprises about 1875 mg myricetin or the derivative or salt thereof and about 300 mg anthocyanin or the derivative or salt thereof.

In some aspects of the method, the ratio of the myricetin or the derivative or salt thereof to the anthocyanin or the derivative or salt thereof is about 1:10-20:1 by weight, or any derivable range therein. In some aspects, the ratio of the myricetin or the derivative or salt thereof to the anthocyanin or the derivative or salt thereof is about 5:1 by weight. In some aspects, the ratio of the myricetin or the derivative or salt thereof to the anthocyanin or the derivative or salt thereof is about 6:25 by weight.

In some aspects of the method, the composition is encapsulated.

In some aspects of the method, the administration is oral administration.

In some aspects, a dosage is administered to the subject in the amount of about 0.5 mg to about 75 mg myricetin or derivative or salt thereof per kg body weight of the subject, or any derivable range therein (e.g., 0.5, 1, 2, 3, 4, 5, 6, 7, 8, 9, 10, 15, 20, 25, 30, 35, 40, 45, 50, 55, 60, 65, 70, or 75 mg), and/or about 0.25 mg to about 50 mg anthocyanin or derivative or salt thereof, or any derivable range therein (e.g., 0.25, 1, 2, 3, 4, 5, 6, 7, 8, 9, 10, 15, 20, 25, 30, 35, 40, 45, or 50 mg), per kg body weight of the subject.

In some aspects, the dosage is administered 3 times/day, 2 times/day, daily, 6/week, 5/week, 4/week, 3/week, 2/week, or 1/week.

In some aspects, the myricetin or the derivative or the salt thereof targets the subject's pancreas, central nervous system, liver, adipose tissue, and/or skeletal muscle. The term “targets” as used herein refers to the capability to bind to a receptor on a tissue or organ and/or to have a physiological effect on a tissue or organ.

In some aspects, the subject has weight loss, preservation of lean muscle mass, preservation of lean muscle mass during weight loss, improved insulin resistance, enhanced mitochondria function, enhanced thermogenesis, decreased plasma lipids, decreased adiposity/lipogenesis, increased metabolic rate, increased browning of white adipose tissue, increased energy expenditure, increased anti-inflammatory effects, improved oxidative stress, and/or the like, and/or any combination thereof after administration of the composition.

In some aspects of the method, the myricetin or the derivative or salt thereof and the anthocyanin or the derivative or salt thereof act synergistically.

In some aspects of the method, the subject is diagnosed as being obese, overweight, and/or suspected of gaining weight. In some aspects, the subject is a juvenile or an adult.

The composition can include one or more high purity active ingredients (e.g., myricetin (or a derivative or salt thereof) or an anthocyanin (or a derivative or salt thereof)), such as at least 75%, 80%, 85%, 90%, 95%, 97%, 98%, 99%, or 99.5% or more pure. The one or more active ingredients can be naturally derived.

The composition or formulation can be stored for one month, 6 months, 12 months, 18 months, or 24 months at room temperature. In some aspects of the invention, the composition is formulated as an edible composition, a dietary supplement, a nutraceutical, a food additive, a food product, a complex carbohydrate, flour, sugar, a beverage, a powder, a tablet, a gel-cap, a bead, an edible tablet or chew (gummy), a gelatin, or a liquid solution for oral administration. In some aspects of the invention, the formulated composition can be comprised in a solid nanoparticle, a lipid-containing nanoparticle, a lipid-based carrier, a sealed conduit, a straw, sealed bag, or any combination thereof.

The compositions of the present invention can also include any one of, any combination of, or all of the following additional ingredients: water, a pharmaceutical agent, a chelating agent, a preservative, a thickening agent, a silicone containing compound, an essential oil, a structuring agent, a vitamin, a pharmaceutical ingredient, or an antioxidant, or a non-naturally occurring compound, or any combination of such ingredients or mixtures of such ingredients. In certain aspects, the composition can include at least two, three, four, five, six, seven, eight, nine, ten, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

In some aspects of the invention, the composition may further comprise one or more carriers or diluents. These carriers/diluents can be adjuvants, excipients, or vehicles such as preserving agents, fillers, disintegrating agents, wetting agents, emulsifiers, suspending agents, sweeteners, flavorings, fragrance, antibacterial agents, antifungal agents, lubricating agents, vitamins, polymers, siloxane containing compounds, essential oils, structuring agents, and dispensing agents. Each carrier is acceptable in the sense of being compatible with the other ingredients of the formulation and not injurious to the subject. In some aspects of the invention, the carrier can include at least one hydrophilic polymeric compound selected from the group consisting of a gum, a cellulose ether, an acrylic resin, a carbohydrate carrier, talc, lactose, mannitol, glucose, water, gelatin, a protein-derived compound, polyvinyl pyrrolidone, magnesium stearate, and any combination thereof. Non-limiting examples of diluents/carriers are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present invention are also contemplated. In certain aspects, the composition is comprised in a container. The container can be a bottle, dispenser, package, or a straw. The container can dispense a predetermined amount of the composition. In certain aspects, the compositions are dispensed as a pill, a tablet, a capsule, an edible chew, a powder, or a liquid. The container can include indicia on its surface. The indicia can be a word, an abbreviation, a picture, or a symbol.

It is contemplated that any embodiment discussed in this specification can be implemented with respect to any method or composition of the invention, and vice versa. Furthermore, compositions of the invention can be used to achieve methods of the invention.

Also contemplated is a product that includes the composition of the present invention. In non-limiting aspects, the product can be a nutraceutical product. The nutraceutical product can be those described in other sections of this specification or those known to a person of skill in the art. Non-limiting examples of products include a pill, a tablet, an edible chew, a capsule, a a gelatin, a spray, a mist, a dissolving film, nutraceutical, a dietary supplement, a food additive, a food product, a complex carbohydrate, flour, sugar, a beverage or a liquid, etc., or any combination thereof.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DETAILED DESCRIPTION OF THE INVENTION

The present invention relates to compositions containing active agents myricetin (or a derivative or salt thereof) or an anthocyanin (or a derivative or salt thereof) (e.g., C3G) or a combination of thereof. Without being bound by theory, in one embodiment, the myricetin can act as a natural GLP-1 agonist that helps reduce caloric intake while an anthocyanin (e.g., C3G) can support the retention of muscle mass by, for example, helping the body burn fat before muscle during caloric output. Therefore, the combination of the two ingredients (e.g., myricetin and C3G) can help reduce weight while maintaining muscle mass in a subject.

Some embodiments of the invention relate to the combined effects of myricetin on GLP-1 receptor activation, insulin secretion, glucose regulation, appetite regulation, adropin elevation, energy homeostasis, mitochondrial function enhancement, thermogenesis, Sirt3 activation, upregulation of UCP1, mitochondrial biogenesis, lipid metabolism modulation, activation of PPARalpha, fatty acid oxidation, suppression of lipogenesis (e.g., via SREBP-1c downregulation), inhibiting dipeptidyl peptidase 4 (DPPV-4) adipose tissue browning, increased energy expenditure, induction of beige adipocytes, activation of β3-adrenergic receptors, anti-inflammatory effects, reduction of oxidative stress, reduction of proinflammatory cytokines, increase in anti-inflammatory adipokines, mitigation of oxidative stress, modulation of neuroendocrine pathways, influence on hypothalamic AMPK pathways, interaction with opioid receptors, and/or the like. Further details on the effects of myricetin can be found in Li, Ying et al. “Myricetin: a potent approach for the treatment of type 2 diabetes as a natural class B GPCR agonist.” FASEB journal: official publication of the Federation of American Societies for Experimental Biology vol. 31, 6 (2017): 2603-2611. doi:10.1096/fj.201601339R; Hu, Tao el all. (2018). Myricetin-induced brown adipose tissue activation prevents obesity and insulin resistance in db/db mice. European Journal of Nutrition. 57. 10.1007/s00394-017-1433-z; Chang C J et al. Myricetin Increases Hepatic Peroxisome Proliferator-Activated Receptor a Protein Expression and Decreases Plasma Lipids and Adiposity in Rats. Evid Based Complement Alternat Med. 2012; 2012:787152. doi: 10.1155/2012/787152. Epub 2012 Mar. 8. PMID: 22474525; PMCID: PMC3310287; Su H M, et al. Myricetin protects against diet-induced obesity and ameliorates oxidative stress in C57BL/6 mice. J Zhejiang Univ Sci B. 2016 June; 17 (6): 437-46. doi: 10.1631/jzus.B1600074. PMID: 27256677; PMCID: PMC4913792; Choi, Ha-Neul et al. “Ameliorative effect of myricetin on insulin resistance in mice fed a high-fat, high-sucrose diet.” Nutrition research and practice vol. 8, 5 (2014): 544-9. doi:10.4162/nrp.2014.8.5.544; and Li, Ying-Xiao et al. “Myricetin Increases Circulating Adropin Level after Activation of Glucagon-like Peptide 1 (GLP-1) Receptor in Type-1 Diabetic Rats.” Pharmaceuticals (Basel, Switzerland) vol. 15, 2 173. 31 Jan. 2022, doi: 10.3390/ph15020173; Akindehin, Seun et al. (2018). Myricetin Exerts Anti-Obesity Effects through Upregulation of SIRT3 in Adipose Tissue. Nutrients. 10. 1962. 10.3390/nu10121962; each of which is incorporated by reference in its entirety.

### I. Definitions

For the purposes of the present disclosure, the following terms have the following meanings:

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The phrase “naturally derived” when referring to a compound/substance means a compound/substance that is obtained from a naturally occurring source. A naturally occurring source is one that is found in nature. This term can be used interchangeably with “natural.” The term “plant-based” when referring to a compound/substance means a compound/substance that is obtained from a plant/tree.

“Derivative,” in relation to a parent compound, refers to a chemically modified parent compound or an analogue thereof, wherein at least one substituent is not present in the parent compound or an analogue thereof. One such non-limiting example is a parent compound which has been covalently modified. Typical modifications are amides, carbohydrates, alkyl groups, acyl groups, esters, pegylations and the like.

“Salt” refers to a salt of a compound that does not cause significant irritation to the organism to which it is administered and does not eliminate the biological activity and properties of the compound. In some embodiments, the salt is an acid addition salt of the compound. Therapeutic salts can be obtained by reacting compounds with inorganic acids such as hydrohalic acid (for example, hydrochloric acid or hydrobromic acid), sulfuric acid, nitric acid, and phosphoric acid. Therapeutic salts can also be obtained by reacting compounds with organic acids, such as aliphatic or aromatic carboxylic acids or sulfonic acids, such as formic acid, acetic acid, succinic acid, lactic acid, malic acid, tartaric acid, citric acid, ascorbic acid, tobacco alkaline acid, methanesulfonic acid, ethanesulfonic acid, p-toluenesulfonic acid, salicylic acid or naphthalenesulfonic acid. Therapeutic salts can also be obtained by reacting compounds with bases to form salts, such as ammonium salts; alkali metal salts, such as sodium or potassium salts; alkaline earth metal salts, such as calcium or magnesium salts; organic bases such as dicyclohexylamine, N-methyl-D-glucosamine, ginseng (hydroxymethyl)methylamine, C 1-C 7 alkylamine, cyclohexylamine, triethanolamine, ethylenediamine) salts; and with amino acids (Such as arginine and lysine) salts.

“Bioavailability” refers to the extent to which the therapeutic agent, such as myricetin (or a derivative or salt thereof) or an anthocyanin (or a derivative or salt thereof), is absorbed from the formulation.

“Systemic,” with respect to delivery or administration of a therapeutic agent, such as a myricetin (or a derivative or salt thereof) or an anthocyanin (or a derivative or salt thereof), to a subject, that therapeutic agent is detectable at a biologically significant level in the blood plasma of the subject.

“Controlled release” or “modified release” refers to the release of the therapeutic agent at such a rate that blood (e.g., plasma) concentrations are maintained within the therapeutic range, but below toxic concentrations over a period of time of about one hour or longer, preferably 12 hours or longer.

The terms “therapeutic agent” or “active agent” or “therapeutic ingredient” or “active ingredient” and their variations refer to an agent/compound/ingredient that has a therapeutic effect. In the present disclosure, a therapeutic agent encompasses myricetin (or a derivative or salt thereof) or an anthocyanin (or a derivative or salt thereof) and others. It also encompasses such compounds together with pharmaceutically acceptable salts thereof. Useful salts are known to those skilled in the art and include salts with inorganic acids, organic acids, inorganic bases, or organic bases. Therapeutic agents useful in the present invention are those compounds that affect a desired, beneficial, and often pharmacological, effect upon administration to a human or an animal, whether alone or in combination with other pharmaceutical excipients or inert ingredients.

The term “therapeutic effect” is art-recognized and refers to a local or systemic effect in animals, particularly mammals, and more particularly humans caused by an active substance. The phrase “therapeutically effective amount” means that amount of such a substance that produces some desired local or systemic effect at a reasonable benefit/risk ratio applicable to any treatment. The therapeutically effective amount of such substance will vary depending upon the subject and disease or condition being treated, the weight and age of the subject, the severity of the disease or condition, the manner of administration and the like, which can readily be determined by one or ordinary skill in the art. For example, certain compositions described herein may be administered in a sufficient amount to produce a desired effect at a reason able benefit/risk ratio applicable to such treatment.

The term “nutraceutical” is recognized in the art and is intended to describe specific chemical compounds found in foods that can prevent disease or ameliorate an undesirable condition. A nutraceutical can be in the form of a dietary supplement, a nutraceutical, a food additive, a food product, a complex carbohydrate, flour, sugar, a beverage, etc., or any combination thereof.

“Patient,” “subject,” or “individual” refers to a mammal (e.g., human, primate, dog, cat, bovine, ovine, porcine, equine, mouse, rate, hamster, rabbit, or guinea pig). In particular aspects, the patient, subject, or individual is a human.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

The term “pharmaceutically acceptable carrier” refers to a pharmaceutically acceptable solvent, suspending agent, or vehicle for delivering a therapeutic agent to a human or animal. The carrier may be liquid, semisolid, or solid.

The term “treatment” or “treating” or “preventing” as used herein means the management and care of a patient for the purpose of combating a condition, such as a disease or a disorder. In one embodiment the term “treatment” or “treating” is intended to include the full spectrum of treatments for a given condition from which the patient is suffering, such as administration of the active compound to alleviate the symptoms or complications; to delay the progression of the disease, disorder, or condition; to alleviate or relieve the symptoms and complications; and/or, to cure or eliminate the disease, disorder, or condition as well as to prevent the condition. In one embodiment prevention is to be understood as the management and care of a patient for the purpose of combating the disease, condition, or disorder and includes the administration of the active compounds to prevent the onset of the symptoms or complications.

The terms “decreasing” or “reducing” or “inhibiting” or any variation of these terms refers to decreasing the quantity referred to by any measurable amount on a daily basis, or over a period of days, weeks, or months, e.g., over 1, 2, 3, 4, 5, 6, 7, 8, 9, 10, or more weeks, or over 1, 2, 3, 4, 5, 6, 7, 8, 9, 10, or more months. For example, a value is decreased or reduced when the value is decreased by at least 1%, 2%, 3%, 4, %, 5%, 6%, 7%, 8%, 9%, 10%, 15%, 20%, 25%, 30%, 35%, 40%, 45%, 50%, or more as compared to the value prior to treatment with any of the disclosed compositions. “Inhibiting” or “reducing” or any variation of these terms includes any measurable decrease or complete inhibition to achieve a desired result.

The terms “maintaining” or “preserving” or “minimizing” or any variation of these terms refers to substantially maintaining the value referred to on a daily basis, or over a period of days, weeks, or months, e.g., over 1, 2, 3, 4, 5, 6, 7, 8, 9, 10, or more weeks, or over 1, 2, 3, 4, 5, 6, 7, 8, 9, 10, or more months. For example, in some embodiments, a value is maintained when the value changes by less than about 15%, 10%, 9%, 8%, 7%, 6%, 5%, 4%, 3%, 2%, or 1% or less as compared to the value prior to treatment with any of the disclosed compositions.

The terms “patient,” “subject,” and “individual” interchangeably refer to a mammal, for example, a human or a non-human mammal, e.g., a primate, dog, cat, bovine, ovine, porcine, equine, mouse, rat, hamster, rabbit, or guinea pig. The term “mammal” or “mammalian” includes murine (e.g., rats, mice) mammals, rabbits, cats, dogs, pigs, and primates (e.g., monkey, apes, humans). In particular aspects in the context of the present invention, the mammal can be a murine mammal or a human.

Generally, “obesity” can be defined as an individual having a body mass index (BMI) of greater than 30 and “overweight” can be defined as an individual having a BMI between 25 and 30. A subject “suspected of gaining weight” refers to, for example, a subject diagnosed with a disorder or condition associated with weight gain over a period of time. A non-limiting example of this is a juvenile diagnosed with childhood obesity.

Also disclosed in the context of the present invention are aspects 1 to 55. Aspect 1 includes a composition comprising therapeutically effective amounts of myricetin or a derivative or salt thereof and an anthocyanin or a derivative or salt thereof.

Aspect 2 is the composition of aspect 1, wherein the anthocyanin or a derivative or salt thereof is cyanidin 3-glucoside, and/or wherein myricetin and the anthocyanin or a derivative or salt thereof is present in the composition at a weight ratio of 8:1 to 3:1, or more preferably about 5:1.

Aspect 3 is the composition of aspect 1 or 2, wherein the composition is capable of activating a MAPK pathway; a PI3K/AKT pathway; a JAK/STAT pathway; a TGF-β pathway; a Wnt/β-catenin pathway; a melaocortin signaling pathway and/or causing one or more of: insulin secretion, glucose regulation, appetite regulation, adropin elevation, energy homeostasis, mitochondrial function enhancement, thermogenesis, Sirt3 activation, upregulation of UCP1, mitochondrial biogenesis, lipid metabolism modulation, activation of PPARalpha, fatty acid oxidation, suppression of lipogenesis (e.g., via SREBP-1c downregulation), inhibiting dipeptidyl peptidase 4 (DPPV-4), adipose tissue browning, increased energy expenditure, induction of beige adipocytes, activation of β3-adrenergic receptors, anti-inflammatory effects, reduction of oxidative stress, reduction of proinflammatory cytokines, increase in anti-inflammatory adipokines, mitigation of oxidative stress, modulation of neuroendocrine pathways, influence on hypothalamic AMPK pathways, interaction with opioid receptors, and/or the like.

Aspect 4 is the composition of any one of aspects 1-3, wherein the composition is capable of activating a GLP-1 receptor.

Aspect 5 is the composition of any one of aspects 1 or 3-4, wherein the anthocyanin is cyanidin 3-glucoside or cyanidin 3-O glucoside.

Aspect 6. is the composition of any one of aspects 1-5, wherein the composition comprises about 100 mg to about 5000 mg myricetin or derivative or salt thereof and about 50 mg to about 3000 mg anthocyanin or derivative or salt thereof.

Aspect 7 is the composition of any one of aspects 1-6, wherein the composition comprises about 1500 mg myricetin or the derivative or salt thereof and about 300 mg anthocyanin or the derivative or salt thereof.

Aspect 8 is the composition of any one of aspects 1-7, wherein the ratio of myricetin or the derivative or salt thereof to anthocyanin or the derivative or salt thereof is about 1:10 to about 20:1 by weight.

Aspect 9. is the composition of any one of aspects 1-8, wherein the ratio of myricetin or the derivative or salt thereof to anthocyanin or the derivative or salt thereof is about 5:1 by weight.

Aspect 10 is the composition of any one of aspects 1-9, wherein the composition is encapsulated.

Aspect 11 is the composition of any one of aspects 1-10, wherein the composition is in oral dosage form.

Aspect 12 is the composition of aspect 11, wherein the oral dosage form is a capsule, a tablet, a caplet, a gel caplet (gelcap), a syrup, a liquid, or a powder, or any combination thereof.

Aspect 13 is the composition of aspect 11 or 12, wherein the oral dosage form is a chewable form, a swallowable form, a dissolvable form, an effervescent, a granulated form, or an oral liquid solution.

Aspect 14 is the composition of any one of aspects 1-13, wherein the composition is in modified-release form.

Aspect 15 is the composition of any one of aspects 1-14, wherein the composition is for weight management, treatment of a metabolic disorder, and/or treatment of an addiction in a subject.

Aspect 16 is the composition of aspect 15, wherein the weight management comprises one or more of weight loss, maintenance of weight loss, preserving lean body mass, minimizing loss of lean body mass during weight loss, decreased food consumption, increasing satiety, reducing pre-meal hunger, reducing intra-meal food intake, or prevention of weight gain in the subject.

Aspect 17 is the composition of aspect 16, wherein the metabolic disorder is selected from is diabetes, a diabetes-related disorder, obesity, or an obesity-related disorder.

Aspect 18 is the composition of any one of aspects 1-17, wherein the myricetin or a derivative or salt thereof or the anthocyanin or a derivative or salt thereof is more than 80% pure.

Aspect 19 is the composition of any one of aspects 14-16 wherein the subject is a juvenile or an adult.

Aspect 20 is the composition of any one of aspects 1-19, wherein the subject is obese, overweight, or suspected to gain weight.

Aspect 21 is the composition of aspect 16, wherein the addiction is an opioid addiction or an alcohol addiction.

Aspect 22 is the composition of any one of aspects 1-21, wherein the myricetin or the derivative or salt thereof and the anthocyanin or the derivative or salt thereof act synergistically.

Aspect 23 is the composition of any one of aspects 1-22, wherein the composition does not include quercetin and/or chlorogenic acid.

Aspect 24 is the dietary supplement comprising the composition of any one of aspects 1-23 in a consumable carrier.

Aspect 25 is the dietary supplement of aspects 24, wherein the dietary supplement is at least 75% natural and/or plant-based.

Aspect 26 is the dietary supplement of aspects 25, wherein the dietary supplement is 100% natural and/or plant-based.

Aspect 27 is the pharmaceutical composition comprising the composition of any one of aspects 1-22 in a pharmaceutically acceptable carrier.

Aspect 28 is the food additive comprising the composition of any one of aspects 1-23.

Aspect 29 is the food product comprising the composition of any one of aspects 1-23 comprising a consumable carrier.

Aspect 30 is the dietary supplement of any one of aspects 24-26, food additive of aspect 28 or the food product of aspect 29, further comprising natural dietary fiber.

Aspect 31 is the dietary supplement, food additive or the food product of aspect 30, wherein the dietary fiber contributes to the bioavailability of the composition.

Aspect 32. is the dietary supplement, food additive or the food product of aspect 30 or 31, wherein the dietary fiber is indigestible dextrin, polydextrose, soybean-derived water-soluble dietary fiber, hydrolyzed guar gum, glucomannan, inulin, pectin, or sodium alginate, or a combination thereof.

Aspect 33 is the food product of aspect 29, wherein the consumable carrier is a cookie, a brownie, a cracker, a breakfast bar, an energy bar, cereal, cake, or beverage.

Aspect 34 is the method for managing weight and/or maintaining cardiovascular health and/or treating a metabolic disorder and/or addiction in a subject, the method comprising administering the composition of any one of aspects 1-23, the dietary supplement of any one of aspects 24-26 or 30-32, the pharmaceutical composition of aspect 27, the food additive of any one of aspects 28 or 30-32 or the food product of any one of aspects 29-33 to the subject.

Aspect 35 is the method of aspect 34, wherein the managing weight comprises one or more of weight loss, maintenance of weight loss, preserving lean body mass, minimizing loss of lean body mass during weight loss, decreased food consumption, increasing satiety, reducing pre-meal hunger, reducing intra-meal food intake, preventing weight gain, or minimizing weight gain.

Aspect 36 is the method of aspect 34 or 35, wherein the metabolic disorder is diabetes, a diabetes-related disorder, obesity, or an obesity-related disorder.

Aspect 37 is the method of aspect 36, wherein: (i) the diabetes related disorder is insulin resistance, glucose intolerance, increased fasting glucose, pre-diabetes, type 1 diabetes, type 2 diabetes, gestational diabetes, hypertension, dyslipidemia, fatty liver disease or a combination thereof; (ii) the diabetes-related disorder is atherosclerosis, arteriosclerosis, coronary heart disease, peripheral artery disease and stroke; or is associated with atherogenic dyslipidemia, blood fat disorders, elevated blood pressure, hypertension, a prothrombotic state, or a proinflammatory state, or a combination thereof; or (iii) the obesity related disorder is selected from obesity linked inflammation, obesity linked gallbladder disease or obesity induced sleep apnea.

Aspect 38 is the method of aspect 34, wherein the addiction is alcoholism or addiction to opioids.

Aspect 39 is the method of any one of aspects 34-38, wherein the anthocyanin is cyanidin 3-glucoside or cyanidin 3-O glucoside.

Aspect 40 is the method of any one of aspects 34-39, wherein the composition comprises about 100 mg to about 5000 mg myricetin or the derivative or salt thereof and about 50 mg to about 3000 mg anthocyanin or the derivative or salt thereof.

Aspect 41 is the method of any one of aspects 34-40, wherein the composition comprises about 1500 mg myricetin or the derivative or salt thereof and about 300 mg anthocyanin or the derivative or salt thereof.

Aspect 42 is the method of any one of aspects 34-41, wherein the ratio of myricetin or the derivative or salt thereof to anthocyanin or the derivative or salt thereof is about 1:10-20:1 by weight.

Aspect 43 is the method of any one of aspects 34-42, wherein the ratio of myricetin or the derivative or salt thereof to anthocyanin or the derivative or salt thereof is about 5:1 by weight.

Aspect 44 is the method of any one of aspects 34-43, wherein the composition is encapsulated.

Aspect 45 is the method of any one of aspects 34-44, wherein the administration is oral administration.

Aspect 46 is the method of any one of aspects 34-45, wherein a dosage administered to the subject in the amount of about 0.5 mg to about 75 mg myricetin or derivative or salt thereof per kg body weight of the subject and/or about 0.25 mg to about 50 mg anthocyanin or derivative or salt thereof per kg body weight of the subject

Aspect 47 is the method of aspect 46, wherein the dosage is administered 3 times/day, 2 times/day, daily, 6/week, 5/week, 4/week, 3/week, 2/week, or 1/week.

Aspect 48 is the method of any one of aspects 34-47, wherein the myricetin or the derivative or the salt thereof targets the subject's pancreas, central nervous system, liver, adipose tissue, and/or skeletal muscle.

Aspect 49 is the method of any one of aspects 34-48, wherein the subject has weight loss, preservation of lean muscle mass, preservation of lean muscle mass during weight loss, improved insulin resistance, enhanced mitochondria function, enhanced thermogenesis, decreased plasma lipids, decreased adiposity/lipogenesis, increased metabolic rate, increased browning of white adipose tissue, increased energy expenditure, increased anti-inflammatory effects, and/or improved oxidative stress after administration of the composition.

Aspect 50 is the method of any one of aspects 34-49, wherein the myricetin or the derivative or salt thereof and anthocyanin or the derivative or salt thereof act synergistically.

Aspect 51 is the method for activating a glucagon-like peptide-1 receptor in a subject, the method comprising administering the composition of any one of aspects 1-23 to the subject, wherein the glucagon-like peptide-1 receptor in the subject is activated.

Aspect 52 is the method of aspect 51, wherein insulin production in the subject is increased after administration of the composition to the subject.

Aspect 53 is the method of reducing weight or preventing/minimizing weight gain of a subject, the method comprising administering the composition of any one of aspects 1-23 to the subject, wherein the subject's weight is reduced or weight gain in a subject is prevented/minimized.

Aspect 54 is the method of aspect 53, wherein the subject is diagnosed as being obese overweight, and/or suspected of gaining weight.

Aspect 55 is the method of aspect 53 or 54, wherein the subject is a juvenile or an adult.

### II. Active Ingredients

As explained above, compositions of the present invention can include myricetin or a derivative or salt thereof or an anthocyanin or a derivative or salt thereof or combinations thereof. The terms “active ingredient” or “active agent” can be used interchangeably with “therapeutic agent,” which refers to compounds that affect a desired, beneficial, and often pharmacological, effect upon administration to a human or an animal, whether alone or in combination with other pharmaceutical excipients or inert ingredients. Myricetin (or a derivative or salt thereof) or an anthocyanin (or a derivative or salt thereof) can be active agents of the invention, used alone or in combination with each other and/or other active agents. In one particular aspect, it was discovered that the combination of myricetin and the anthocyanin cyanidin 3-glucoside (C3G) can be particularly effective in reducing a person's weight and/or limiting muscle loss, and/or a weight ratio of myricetin to C3G at 10:1 to 1:10 or any range therein (e.g., 10:1 to 1:10, 9:1 to 1:9, 8:1 to 1:8, 7:1 to 1:7, 6:1 to 1:6, 5:1 to 1:5, 4:1 to 1:4, 3:1 to 1:3, 2:1 to 1:2, or 1:1), or preferably 7:1 to 3:1, or more preferably about 6:25 or 5:1.

### A. Myricetin

Myricetin is a naturally occurring flavonoid with a wide range of biological properties. Its chemical structure is characterized by a three-ring backbone, with multiple hydroxyl (OH) groups, particularly prominent on the B ring. These hydroxyl groups can contribute to its antioxidant properties and ability to scavenge free radicals. Myricetin is primarily found in various fruits, vegetables, teas, and medicinal plants, where it plays a role in protecting the plants from environmental challenges including oxidative stress. Common dietary sources of myricetin include berries, red onions, walnuts, tea leaves, grapes and various herbs. Certain plants and trees, such as Myrica species (e.g., Myrica rubra), and other members of the Myricaceae family, are especially high in myricetin.

To obtain myricetin, plant materials with high flavonoid content, such as leaves from Myrica species, can be carefully selected and harvested at peak growth stages to maximize yield. After harvesting, the plant material can be dried and ground into a fine powder, which then undergoes solvent extraction, often with an ethanol and water mixture to effectively extract the flavonoids. The resulting crude extract can be filtered to remove large particles, and further purification can be achieved through liquid-liquid extraction, where solvents selectively isolate myricetin from other flavonoids and impurities. Chromatography techniques, particularly high-performance liquid chromatography (HPLC), can then be applied to refine the myricetin further, producing a highly purified compound often exceeding 95% purity

Once isolated, the purified myricetin is typically crystallized and carefully dried to eliminate any remaining solvents, resulting in a stable powdered form. This high-purity myricetin can undergo rigorous quality testing, including mass spectrometry, HPLC, and nuclear magnetic resonance (NMR) spectroscopy, to confirm its chemical structure and purity level. After quality validation, the purified myricetin can be packaged and stored in a dark, cool, and dry environment to prevent degradation, preserving its integrity for laboratory studies and pharmacological research. Other methods of obtaining myrecetin are known in the art.

Myricetin can have the following chemical structure:

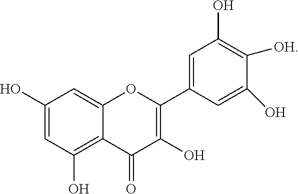 [SMILES: O=c1c(O)c(-c2cc(O)c(O)c(O)c2)oc2cc(O)cc(O)c12]

Derivatives of myricetin can include, for example, additional OH groups, removal of OH groups, esters of the OH groups (e.g., alkyl group bonded to the oxygen atom and removal of the hydrogen atom from the oxygen atom). Some examples of derivatives can include quercetin, kaempferol, apigenin, mericitrin, catectin, epicatechin gallate (ECG), and epigallocatechin-3-gallate (EGCG). Some additional examples of myricetin derivatives can include 3,7, 4′, 5′-tetramethyl ether of myricetin, myricetin 3-O-rutinoside, myricetin 3-O-rhamnoside, myricetin 3-O-glucuronide, myricetin 3-O-galactoside, myricetin 3-O-glucoside, myricetin 3-O-xylo-pyranoside, myricetin 3-O-arabino-pyranoside, and myricetin 3-O-arabino-furanoside. Combinations of myricetin with myricetin derivatives or combinations of myricetin derivatives is also contemplated in the context of the present invention.

### B. Cyanidin 3-Glucoside (C3G)

Cyanidin-3-glucoside (C3G) is a naturally occurring anthocyanin, a type of flavonoid pigment responsible for the red, purple, and blue colors in many fruits and vegetables. Its structure consists of a cyanidin molecule (a three ring flavonoid skeleton with multiple hydroxyl (—OH) groups on its B ring, typically at the 3′ and 4′ positions, and another on the C ring at the 3 position) bound to a glucose sugar, which enhances its solubility and stability in plant tissues. Cyanidin-3-glucoside is found in deeply colored fruits and vegetables, particularly those with red, purple, and blue pigments. Common dietary sources of C3G include black rice, berries (blackberries, blueberries, black currants, elderberries), red cabbage, purple sweet potatoes and cherries. The high concentration of C3G in these sources can contribute to their antioxidant capacity and offer a natural means of protection against environmental stressors.

C3G can have the following chemical structure:

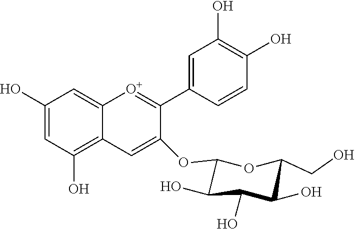

In some aspects, salts of C3G can be formed with the O⁺ group (e.g., a negative ion (e.g., Cl⁻) can be used to form a salt of C3G).

To obtain cyanidin-3-glucoside (C3G), the process can begin with harvesting plant materials that are rich in anthocyanins, such as berries or purple sweet potatoes, at peak ripeness when anthocyanin levels are highest. These materials can be processed immediately or frozen to preserve anthocyanin content. After washing, drying, and grinding the material into a fine powder, extraction can be carried out using acidified methanol or ethanol, which enhances the solubility and stability of anthocyanins like C3G. The crude extract can be filtered to remove any solid residues, and then liquid-liquid partitioning can be used to separate the anthocyanin-rich fraction from other plant compounds based on their solubility differences. Other methods of obtaining C3G are known in the art.

### C. Anthocyanin

In addition to C3G, or as replacement of C3G, other anthocyanidins and anthocyanins (anthocyanidins including sugar groups) can be used in the context of the present invention. Anthocyanidins and anthocyanins are a large family of naturally occurring pigments. The color of most fruits, flowers and berries is determined by their content of anthocyanidins and anthocyanins. The term “anthocyanin” as used herein is intended to include both glycosylated anthocyanins (anthocyanosides) as well as the aglycon of the anthocyanoside (anthocyanidin).

Anthocyanin materials can be obtained from plant sources, such as leaves, twigs, bark, roots, stern, seeds, flowers, berries, fruit, for example, by routine isolation methods from suitable plants sources noted, but not limited to, those described herein. There are various methods for the extraction of anthocyanins known to those of skill in the art. Some of these methods are described in, for example, U.S. Pat. Nos. 5,817,354; 5,200,186; 5,912,363; 4,211,577; 4,302,200 (each incorporated herein by reference).

Examples of suitable anthocyanin-containing plants include, but are not limited to, fruits, vegetables, flowers and other plants selected from the group consisting of Acer macrophyllum, Acer platanoides, acerola, Ajuga reptans, apple, apricot, Artict bramble, avocado, banana, barberry, barley, Begonia semperfiorens, Bellis perennis, Bletilla striata, bilberry, black beans, black soybeans, black, blue and purple potatoes, blackberry, blueberry, bog whortleberry, boysenberry, buckwheat, cacao, Camellia sinensis, canarygrass, Caucasian blueberry, Chimonanthus praecox, celery, Cerasus avium, cherry, cherry laurel, chicory, chive, chokeberry, Cornelian cherry, cornflower, cotoneaster, cowberry, cranberry, crowbeny, chrysanthemum, Cynomorium coccineum, Dahlia variabilis, danewort, deerberry, Dendrobium, dwarf dogwood, Echinacea purpea, eggplant, elderberry, fababean, Fatsia japonica, feijoa, fig, garlic, gerbera, ginseng, Globe artichoke, gooseberry, grapes, guava, hawthorn, hibiscus or roselle, Hibiscus Sabdaiffa, highbush blueberry, hollyhock, honeysuckle, Ipomoea purpurea, Iris ensata, Java plum, Jerusalem artichoke, kokum, Laeliocattleya, lentil, loganberry, lupine, lychee, maize, mango, mangosteen, maqui, Matthiola incana, meconopsis, Metrosideros excelsa, millet, mountain ash berry, mulberry, myrtle berry, olive, onion, orange, ornamental cherry, passion fruit, pea, peach, peanut, pear, perilla, petunia, Phalaenopsis, Phalsa, Pharbitis, Pineapple, pistachio, plum, pomegranate, Phragmites australis, purple carrot, quince, rabbiteye blueberry, radish, red and black currant, red and black raspberry, red cabbage, rice, rhubarb, rosehip, rye, saffron, sarracenia, sheepberry, Sophronitis coccinea, sorghum, sparkleberry, strawberry, Fragada Vesca, sugarcane, sunflower, sweet cherry, sweet potato, tamarillo, tamarind, taro, tart cherry, Tulip greigii, turnip, water lily, Weigela, wheat, wild rice, Verbena hybrida, yam and mixtures thereof.

The term “anthocyanin” as used herein is intended to refer not only to monomeric anthocyanins, but also refers to dimeric and polymeric (i.e. containing from 3 to 20 anthocyanidin monomer residues) forms of anthocyanins and to leucoanthocyanidins (also known as flavan-3,4-diols). The anthocyanins can comprise substitutions (e.g. alkyl, alkoxy groups etc.) and in particular can be O-glycosylated.

Anthocyanins that can be useful in the inventions described herein include, but are not limited to, cyanidin-3-glucoside; cyanidin 3-glucosylrutinoside; cyanidin-3-gentibioside; cyanidin-3-rutinoside, cyanidin-3-sambunigrin, cyanidin-3-samb-5-glucoside, cyanidin-3-galactoside, peonidin-3-rutinoside, peonidin-3-glucoside, peonidin-3-galactoside, peonidin, cyanidin, cyanidin-3 sophoroside, pelargonidin, delphinidin, delphinidin-3-glucoside, delphinidin-3-galactoside, petunidin, petunidin-3-glucoside, petunidin-3-galactoside, malvidin, malvidin-3-arabinoside, malvidin-3-glucoside, galactoside, kaempferol, hesperidin, gentiodelphin, platyconin, cinerarin and the like.

Suitable examples of anthocyanins from various plants, include, but are not limited to Acer macrophyllum, Cyanidin derivative, Acer platanoides, Cyanidin 3-(2″,3″-digalloyl-beta-glucopyranose (3%), Cyanidin 3-(2″-galloyl-beta-glucopyranose (37%), Cyanidin 3-beta-glucopyranoside (60%), Acerola, Malpighia marginata, Cyanidin-3-glucoside, Cyanidin-3-glucoside, Ajuga reptans, Cyanidin 3-(di-p-coumaroyl) sophoroside-5-glucoside, Apple, Malus spp, Cyanidin 3-galactoside, Cyanidin 3-galactoside, Cyanidin 3-arabinoside, Cyanidin 3-glucoside, Cyanidin 3arabinoside, Cyanidin 3-xyloside, Cyanidin 3glucoside, Cyanidin 3-xyloside, Apricot, Prunus armeniaca, Cyanidin-3-glucoside, Cyanidin-3glucoside, Artic bramble, Rebus spp, Avocado, Persea spp, Acylated cyanidin 3,5-diglucoside, Cyanidin 3-galactoside, Cyanidin 3-galactoside, Banana, Musa acuminata, M. balbisiana, Barberry, Berberis spp., Cyanidin-glucoside, Cyanidin-glucoside, Barley, Hordeum vulgare, Cyanidin and cyaniding glycosides, Bean, Pheseolus vulgaris (several cultivars), Cyanidin 3-glucoside, Cyanidin 3-glucoside, Cyanidin 3,5-diglucoside, Begonia semperflorens cvs, Cyanidin derivative, Benibana-cha, Camellia sinensis, Cyanidin 3-O-beta-D galactoside, Cyanidin 3-O-beta-D-galactoside, Bellis perennis, 3 Cyanidin 3-derivatives, Bletilla striata, Acylated cyanidin 3,7,3′-triglucoside derivatives, Bilberry, Vaccinium myrtillus, Artemis/Iprona; Indena, Cyanidin-3-galactoside (22%); Cyanidin-3-galactoside, Cyanidin-3-glucoside (9%), Cyanidin-3glucoside, Black beans, Phaseolus, Cyanidin-3-glucoside (96%), Cyanidin-3glucoside, Blackberry (European and American), Moriferi veri, Rubus caesius, R. Alleghniensis, R. argufus, R. cuneifolius, R. setosus, R. trivials, Cyanidin-glucoside (70-100%), Cyanidin-glucoside, Cyanidin-rutinoside, Black grapes, Many varieties, Black potatoes, Solanumtuberosum tuberosum, Cyanidin-glycosides, Black raspberry, Rubus occidentalis, Cyanidin-sambubloside (20%); Cyanidin-sambubloside, Cyanidin-xylosylrutinoside (40%); Cyanidin-glucoside, Cyanidin-glucoside, (17%), Cyanidin-rutinoside (23%), Black soybeans, Glycine max, Cyanidin-3-glucoside (96%), Cyanidin-3-glucoside, Blueberries, Five common Vaccinium spp, Cyanidin-glucoside (3%); Cyanidin-glucoside, Cyanidin-galactoside (3%), Cyanidin galactoside, Cyanidin-arabinoside (3%), Cyanidin-3-arabinoside, Bog whortleberry, Vaccinium uliginosum, Cyanidin-3-glucoside (14%), Cyanidin 3 glucoside (14%), Cyanidin #arabinoside (10%), Cyanidin-3-arabinoside (10%), Cyanidin 3-galactoside (6.5%), Cyanidin-3-galactoside (6.5%), Boysenberry, new Zealand, Cyanidin-3-sophoroside (44.5%), Cyanidin-3-glucoside, Cyanidin-3-glucoside (26.4%), Cyanidin-3 glycosylrutinoside (25.8%), Cyanidin-rutinoside (3.3%), Buckwheat, Fagopyrum species, Cyanidin-3-glucoside, Cyanidin-3-glucoside, Cyanidin 3-galactoside, Cyanidin-3-galactoside, Cacao, Theobroma cacao, Cyanidin 3-glucoside (suspected), Cyanidin-3-glucoside (suspected), Celery, Apium spp, Cherry laurel, Prunus laurocerasus, Cyanidin-3-arabinoside, Cyanidin-3-arabinoside, Chicory, Cichorium intybus, Cyanidin 3-glucoside, Cyanidin 3-glucoside, Chive, Allium schoenoprasum, Cyanidin-3-glucoside, Cyanidin-3-glucoside, Cyanidin-3-acetyl glucoside, Cyanidin 3-(6 malonyl glucoside), Cyanidin 3-(3,6 dimalonylglucoside), Chokeberry, Aronia melanocarpa, Artemis/Iprona, Cyanidin-3-galactoside (64.5%), Cyanidin-3-galactoside, Cyanidin-3-arabinoside (28.9%), Cyanidin-3 arabinoside, Cyanidin-3-glucoside (2.4%), Cyanidin-3 glucoside, Cyanidin-3-xyloside (4.2%), Cyanidin-3-xyloside, Coffee, Coffea arabica cv. Bourbon Vermelho, Cyanadin-3-glycoside, Cyanadin 3,5-diglyeoside, Cyanadin 3-glycoside, Cotoneaster, Cotoneaster Medic. Spp, Cyanidin 3-glucoside, Cyanidin 3-glucoside, Cyanidin 3-galactoside, Cyanidin 3-rutinoside, Cyanidin 3 galactoside, Cowberry or Lingonberry, V. vitis-idaea, Cyanidin 3-galactoside Cyanidin 3-arabinoside, Cyanidin 3-galactoside, Cyanidin 3-glucoside, Cyanidin 3 arabinoside, Cyanidin 3 glucoside, Chimonanthus praecox, Cyanidin 3-O-glucoside, Cyanidin-3-O-glucoside, Acylated cyanidin 3-O-glucoside, Cyanidin glycoside, Cranberry (American and European), Vaccinium macrocorpon, Ocean Spray, Cyanidin-galactoside (16-24%), Cyanidin-galactoside, V. oxycoccus, Cyanidin-arabinoside (13-25%), Cyanidin arabinoside, CrOwberry, Empetrum nigrum, Cyanidin 3-glucoside Cyanidin 3,5-diglucoside, Cyanidin 3-glucoside, Cyanidin 3-rutinoside, Cyanidin 3-sophoroside, Chrysanthemum, Dendranthema Grandiflorum, Cyanidin 3-O-(6′-O-malonyl-beta-glucopyranoside, Currant (red and black), Ribes rubrum, R. nigrum, Cyanidin-glucoside (2-10%), Cyanidin-glucoside, Cyanidin sambubioside, Cyanidin-rutinoside (8-17%), Cyanidin-sambubioside (9-31%), Cyanidin-sophoroside (4-9%), Cyanidin xylosylrutinoside (28-73%), Cyanidin glucosylrutinoside (14-28%), Cyneinonurn coccineum, Cyanidin 3-O-glucoside (92%), Cyanidin 3-O-glucoside (92%), Cyanadin 3-O-(6-O rhamnosylglucoside (8%), Danewort, Sambucus ebulus, Cyanidin 3-xylosylglucoside, Cyanidin 3-sambubioside, Cyanidin 3 sambubloside, Cyanidin 3-glucoside, Cyanidin 3-sambubioside-5-glucoside, Cyanidin 3,5diglucoside, Cyanidin 3-glucoside, Cyanidin 3-arabinoglucoside, Dendrobium, Phalaenapsis spp, Cyanidin derivatives, Dwarf dogwood, Comus suecica, Cyanidin 3-glucoside (4%), Cyannidin 3-glucoside (4%), Cyanidin 3-galactoside (16%), 2 Cyanidin derivatives (80%), Echinacea, Echinacea spp., Eldenberry, Sambucus nigra, Artemis/Iprona, Cyanidin-3-glucoside (42%), Cyanidin-3-glucoside, Cyanidin-3-sambubioside (43%) Cyanidin-3,5-diglucoside (2%), Cyanidin-3 sambubloside-5 glucoside (9%), Gentians spp, Cyanidin 3-O-beta-D-glucoside and 3 other derivatives, Cyanidin 3-O-beta-D-glucoside, Fatsia japonica, Cyanidin 3-lathyroside, Feijoa, Feijoa sellowiana, Cyanidin 3-glucoside, Cyanidin 3-glucoside, Fig, Ficus carica spp, Cyanidin 3-rhamnoglucoside, Cyanidin 3,5-diglucoside, Cyanidin 3-glucoside, Forsythia X, intermedia cv, Spring Glory, Cyanidin derivatives, Garlic, Allium sativum, Cyanidin 3-glucoside, Cyanidin 3-glucoside, Cyanidin 3-glucoside, Cyanidin 3-glucoside monoacylated, Cyanidin 3-glucoside triacylated, Ginseng, Panax ginseng, Panax quinquefolius, Cyanidin 3-O-β-D-xylopyranyl-(1 2)-β-D-glucopyranoside, Globe artichoke, Cynara scolymus, Cyanidin 3-caffeylglucoside, Cyanidin 3-caffeylsophoroside, Cyanidin 3-dicaffeylsophoroside, Gooseberry, Ribes spp, Cyanidin 3-glucoside, Cyanidin 3-glucoside, Cyanidin 3-rutinoside, Grape, Vinis vinifera, Cyanidin 3-monoglucoside, Cyanidin 3-monoglucoside, Cyanidin 3-monoglucoside-acetate, Cyanidin 3-monoglucoside-p-coumarate, Guava, Psidium guajavica, Cyanidin 3-glucoside, Cyanidin 3-glucoside, Hawthorn, Crataegus spp, Cyanidin 3-galactoside, Cyanidin 3-galactoside, Cyanidin 3-arabinoside, Cyanidin 3-glucoside, Cyanidin 3 glucoside, Hibiscus or Roselle, Hibiscus sabdariffa, Cyanidin-sambubioside (30%), Hollyhock, Althaea rosea, Cyanidin 3-glucoside, Cyanidin 3-rutinoside, Cyanidin 3-glucoside, Other cyaniding glucosides, Honeysuckle, Lonicera nitida, Cyanidin 3-rutinoside, Cyanidin 3-glucoside, Cyanidin 3-glucoside, Japanese garden iris, Iris ensata, Cyanidin 3RG, Cyanidin 3RG5G, Cyanidin 3Rgac5G, Ipornoea purpurea, Six acylated cyanidin 3-sophoroside-5 glucosides, Java plum, Mytciana jaboticaba, Cyanidin 3-glucoside, Cyanidin 3-glucoside, Jerusalem artichoke, Helianthus tuberosus, Kokum, Garcinia indica, Cyanidin 3-glucoside, Cyanidin 3-glucoside, Cyanidin 3-sambubioside, Cyanidin 3-sambubioside, Laelioeattleya cv Mini purple, Acylated cyaniding derivatives, Lactuca saliva, Cyanidin 3-O-(6″-malonylglucoside), Loganberry, Rubus loganbaccus, Cyanidin-sophoroside (48.1%), Cyanidin-glucoside, Cyanidin-glucoside (21.6%), Cyanidin-rutinoside (6.2%), Lupine, Lupinus spp, Cyanidin glycosides, presence confirmed, Lychee, Litchi chinensis, Cyanidin 3-glucoside, Cyanidin 3-glucoside, Cyanidin 3-galactoside, Cyanidin 3-rutinoside, Cyanidin 3 galactoside, Maize, Zea mays, Cyanidin 3-glucoside, Cyanidin 3-glucoside, Cyanidin 3-(6″-malonylglucoside) Cyanidin 3 (3″,6″dimalonyl-glucoside) Mango, Mangifera indica, (Cyanidin glycosides, Mangosteen, Garcina mangostana, Cyanidin 3-sophoroside, Cyanidin 3-glucoside, Cyanidin 3-glucoside, Maqul, Aristotella chilensis, Cyanidin 3-,5-diglucoside, Matthiola incana, Four acylated cyaniding 3-sambubloside-5 glucosides, Millet, Pernnisetum americanum, Cyanidin 3-glucoside, Cyanidin 3-glucoside, Mountain ash berry, Sorbus spp, Cyanidin 3-galactoside, Cyanidin 3,5-diglucoside Cyanidin 3-β-D glucopyranoside, Mulberry, Morus nigra, Cyanidin 3-glucoside, Cyanidin 3-glucoside, Cyanidin 3,5-diglucoside, Cyanidin 3-rutinoside, Cyanidin 3-sophoroside, Myrtle berry, Myrtus communis, Cyanidin 3-glucosides, Cyanidin 3-glucosides, Cyanidin 3-diglucoside, Olive, Olea europaea, Cyanidin 3-rutinoside, Cyanidin 3-glucoside, Cyanidin 3-glucoside, Cyanidin derivatives, Onion, Allium sepa, Cyanidin 3-glucoside, Cyanidin 3-glucoside, Cyanidin 3-diglucoside, Cyanidin 3-laminarioside, Orange, Citrus sinensis, Cyanidin 3-glucoside (95%), Cyanidin 3-glucoside, Cyanidin 3,5-diglucoside, Passion fruit, Pasiflora edulis, Cyanidin 3-glucoside, Cyanidin 3-glucoside, Pea, Pisum sativum, Cyanidin 3-sophoroside glucosides, Cyanidin 3-sambubioside-5-glucosides, Peach, Prunus persica, Cyanidin 3-glucoside, Cyanidin 3-glucoside, Cyanidin 3-rutinoside, Cyanidin derivatives, Peanut, Arachis hypogaea, Cyanidin glucosides, Pear, Pyrus communis, Cyanidin 3-galactoside, Cyanidin 3-galactoside, Cyanidin 3-arabinoside, Cyanidin 3-arabinoside, Perilla, Perilla frutescens, Cyanidin 3,5-diglucoside, Cyanidin 3,5-derivatives, Petunia spp, Cyanidin 3-rutinoside, Phalsa, Grewia spp, Cyanidin 3-glucoside, Cyanidin 3-glucoside, Pineapple, Anans comosus, Cyanidin 3-galactoside, Cyanidin 3-galactoside, Pistachio, Pistacia vera, Pragmites australis, Cyanidin-3 derivatives, Plum, 2000 varieties, 15 species, Cyanidin-glucoside (37%), Cyanidin glucoside, Cyanidin-rutinoside (45%), Pomegranate, Punica granatam, Cyanidin-glucoside (30%), Cyanidin-glucoside, Cyanidin-diglucoside (17%), Purple carrot, Daucus carota, Cyanidin-glucoside, Cyanidin-glucoside, Cyanidin-glucosylgalactoside, Cyanidin-galactoside, Cyanidin-digalactoside, Cyanidin-galactoside, Quince, Cydonia oblonga, Cyanidin-3 glucoside, Cyanidin 3,5-diglucoside, Cyanidin derivatives, Radish, Raphanus sativus, Acylated cyanidin 3-sophoroside-5-glucoside, Acylated cyanidin 3 diglucoside-5-glucoside, Red cabbage, Brassica oleracea var capitata, Cyanidin glycosides, Reed, Phalaris arundinacea, Cyanidin 3-glucoside, Cyanidin 3-glucoside, Cyanidin 3-(6″-malonylglucoside), Cyanidin 3 (3″,6″dimalonyl-glucoside), Red onion, Allium cepa, Cyanidin 3-glucoside, Cyanidin 3-glucoside, Acylated cyanidin 3-glucoside derivatives, Red petunia, Petunia spp, Cyanidin 3-glucoside, Cyanidin 3-glucoside, Cyanidin 3-sophoroside, Red raspberry, Rubus idaeus, Cyanidin glucoside (17%), Cyanidin-glucoside, Cyanidin-rutinoside (7%), Cyanidin-sophoroside (50%), Cyanidin glycosylrutinoside (26%), Cyanidin-diglucoside, Rhubarb, Rneum spp, Cyanidin 3-glucoside, Cyanidin 3-glucoside, Cyanidin 3-rutinoside, Rice, Oryza spp, Cyanidin 3-glucoside, Cyanidin 3-glucoside, Cyanidin 3-rhamnoside, Cyanidin 3,5-diglucoside, Rosehip, Rosa canina, Cyanidin 3-rutinoside, Cyanidin 3-glucoside, Cyanidin 3-glucoside, Cyanidin 3,5-diglucoside, Rye, Secale cereale, Cyanidin 3-glucoside, Cyanidin 3-glucoside, Cyanidin 3-rhamnosylglucoside, Cyanidin 3-rhamnosyldiglucoside, Cyanidin 3-rutinoside, Cyanidin 3-rutinoside derivatives, Cyanidin 3-gentiobioside, Sheepberry, Viburnum spp, Cyanidin 3-glucoside, Cyanidin 3-glucoside, Cyanidin 3-arabinosylsambubioside, Sorghum, Sorghum bicolor, Cyanidin, Cyanidin glycosides, Sparkleberry, V′ arboreum, Cyanidin 3-glucoside, Cyanidin 3-glucoside, Cyanidin 3-arabinoside, Cyanidin 3-galactoside, Strawberry, Fragaria ananassa, Cyanidin-glucoside (minor), Cyanidin-glucoside, Sunflower, Hellanthus annuus, Cyanidin 3-glucoside, Cyanidin 3-glucoside, Acylated cyanidin 3-glucoside, Cyanidin 3-xyloside, Cyanidin 3-xyloside, Acylated cyanidin 3-xyloside, Cyanidin 3-vanillyl sambubioside, Sweet cherry, Prunus avintn, Cyanidin-glucoside, Cyanidin-glucoside, Cyanidin-rutinoside; Cyanidin 3-suphoroside, Sweet potato, Ipornoea batatas Sophronitis coccinea, Cyanidin derivatives, Five acylated cyanidin 3,3′,7-triglucosides, Tamarillo or tomato tree, Cyphomandrea betacea, Cyanidin 3-rutinoside, Cyanidin 3-glucoside, Cyanidin 3-glucoside, Tamarind, Tamarindus indica, Cyanidin 3-glucoside, Cyanidin 3-glucoside, Taro, Colocasia esculenta, Cyanidin 3-glucoside, Cyanidin 3-glucoside, Cyanidin 3-rutinoside, Tart Cherry (balaton), Prunus cerasus cv. Balaton, Nutrilite, Cyanidin-3-rutinoside-hexose (75%), Cyanidin-3-rutinoside-pentose (3%), Cyanidin-3-rutinoside (18%), Tart cherry (montmorency), Prunus cerasus cv. Montmorency, Nutrilite, Cyanidin-3-sophoroside (80%), Cyanidin-3-glucoside (20%), Cyanidin-3-glucoside (20%), Tulip, Tulipa spp, Cyanidin 3-O-(6″-rhamnosylglucosides), Cyanidin 3-O-derivative, Turnip, brissica rapa, Cyanidin 3-glucoside, Cyanidin 3-glucoside, Cyanidin 3-diglucoside-5-glucoside, Water lily, Nymphasa alba, Cyanidin 3-O-(6″-acetyl-beta-galactopyrosinase (23%), Cyanidin 3-O-galactoside (2%), Cyanidin 3-O-galactoside (2%), Weigela spp, Cyanidin 3-O-glucoside, Cyanidin 3-O-glucoside, Cyanidin 3-O-glucoside xylose, Wheat, Triticum spp, Cyanidin 3-glucoside, Cyanidin 3-glucoside, Acylated cyanidin glucoside, Cyanidin 3-rutinoside, Acylated cyanidin 3-rutinoside, Cyanidin 3-gentiobioside, Wild rice, Zizania aquatica, Cyanidin 3-glucoside, Cyanidin 3-glucoside, Cyanidin 3-rhamnoglucoside, and Yam, Dioscoracea spp, Cyanidin 3,5-diglucoside, Cyanidin 3-glucoside, Cyanidin 3-glucoside, Cyanidin 3-rhamnoglucoside, Cyanidin 3-gentiobioside, Acylated cyanidin glucosides.

The anthocyanin in the composition can be a single anthocyanin, or comprise a mixture of anthocyanins. In particular, the anthocyanin is selected from the group consisting of malvidin, cyanidin, delphinidin, paeonidin, pelargonidin and petunidin, and glycosides thereof. The anthocyanin can be commercially available in a purified form. Alternatively, the anthocyanin can be obtained by extracting anthocyanin containing plants such as grape, black carrot, red cabbage, blackberry, blackcurrent, cranberry and the like as described above.

### III. Compositions

As described above, the compositions of the present invention can comprise therapeutically effective amounts of myricetin or a derivative or salt thereof and an anthocyanin or a derivative or salt thereof. In some embodiments the anthocyanin is C3G or cyanidin 3-O glucoside.

### A. Cellular Mechanisms

The composition can be capable of activating cellular pathways, such as metabolic pathways. Exemplary metabolic pathways include, but are not limited to, a MAPK pathway, PI3K/AKT pathway, JAK/STAT pathway, TGF-β pathway, AMPK pathway, Wnt/β-catenin pathway, melaocortin signaling pathway, and/or the like, and/or any signaling pathway or receptor activation related to weight management, metabolic disorders, and/or addiction. Exemplary metabolic pathways and receptors are provided in Wen, X., Zhang, B., Wu, B. et al. Signaling pathways in obesity: mechanisms and therapeutic interventions. Sig Transduct Target Ther 7, 298 (2022), which is incorporated by reference in its entirety herein.

In some embodiments of the composition acts as a GLP-1 receptor agonist. In some embodiments, the composition acts as an agonist on one ore more receptors associated with metabolism, metabolic diseases and/or addiction in addition or instead of GLP-1 receptor.

### B. Concentrations and Ratios

In some embodiments, the myricetin or the derivative or salt thereof is present in the composition (for example in a single dose or serving) in an amount ranging from about 100 mg to about 5000 mg (e.g., about 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1550, 1600, 1650, 1700, 1750, 1800, 1850, 1875, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, mg) or any derivable range therein. In some embodiments, myricetin or the derivative or salt thereof is present in the composition (for example in a single dose or serving) in an amount ranging from about 500 mg to about 3000 mg, from about 750 mg to about 2500 mg, from about 1000 mg to about 2000 mg, or from about 1250 mg to about 1750 mg. In some embodiments, the amount of myricetin or the derivative or salt thereof is present in the composition (for example in a single dose or serving) at about 1500 mg. In some embodiments, the amount of myricetin or the derivative or salt thereof is present in the composition (for example in a single dose or serving) at about 1875.

In some embodiments, the anthocyanin or the derivative or salt thereof is present in the composition (for example in a single dose or serving) in an amount ranging from about 50 mg to about 3000 mg (e.g., about 50, 55, 60, 65, 70, 75, 80, 85, 90, 95, 100, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1550, 1600, 1650, 1700, 1750, 1800, 1850, 1875, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000 mg) or any derivable range therein. In some embodiments, the anthocyanin or the derivative or salt thereof is present in the composition (for example in a single dose or serving) in an amount ranging from about 50 mg to about 500 mg, from about 100 mg to about 400 mg, from about 150 mg to about 450 mg, or from about 250 mg to about 350 mg. In some embodiments, the amount of anthocyanin or the derivative or salt thereof is present in the composition (for example in a single dose or serving) at about 300 mg.

The ratio of myricetin (or the derivative or salt thereof) to anthocyanin (or the derivative or salt thereof) can be about 0.5:1.0 (w: w) to about 20.0:1.0 (w: w) (e.g., about 0.1:1.0, 0.5:1.0, 1.0, 1.5:1.0, 2.0:1.0, 2.5:1.0, 3.0:1.0, 3.5:1.0, 4.0:1.0, 4.5:1.0, 5.0:1.0, 5.5:1.0, 6.0:1.0, 6.5:1.0, 7.0:1.0, 7.5:1.0, 8.0:1.0, 8.5:1.0, 9.0:1.0, 9.5:1.0, 10.0:1.0, 10.5:1.0, 11:1.0, 11.5:1.0, 12:1.0, 12.5:1.0, 13:1.0, 13.5:1.0, 14:1.0, 14.5:1.0, 15:1.0, 15.5:1.0, 16:1.0, 16.5:1.0, 17:1.0, 17.5:1.0, 18:1.0, 18.5:1.0, 19:1.0, 19.5:1.0, or 20:1.0). In some embodiments, the ratio range in by weight can be from about 0.1:1.0 to about 15:1, from about 0.5:1.0 to about 10:1, from about 1:1: to about 9:1, from about 3:1 to about 7:1, from about 3.5:1 to about 1.6.5:1, from about 4:1 to about 6:1, or from about 4.5:1 to about 5.5:1 myricetin (or the derivative or salt thereof) to anthocyanin (or the derivative or salt thereof). 1: In some embodiments, the ratio of myricetin (or the derivative or salt thereof) to anthocyanin (or the derivative or salt thereof) is about 5:1 by weight.

In some embodiments, the ratio range can be from about 8.25:1 to about 4.25:1, from about 8:1 to about 4.5:1, from about 7.75:1 to about 4.75:1, from about 7.5:1 to about 5:1, from about 7.25:1 to about 5.25:1, from about 7:1 to about 5.5:1, from about 6.75:1 to about 5.75:1, from about 6.5:1 to about 6:1, or 6.25:1. In some embodiments, the ratio of myricetin (or the derivative or salt thereof) to anthocyanin (or the derivative or salt thereof) is about 6.25 by weight.

### IV. Composition Vehicles

The compositions of the present invention can be incorporated into all types of vehicles. Non-limiting examples include dietary supplement, a nutraceutical, a food additive, a food product, a complex carbohydrate, flour, sugar, a beverage, and any combination thereof; emulsions (e.g., water-in-oil, water-in-oil-in-water, oil-in-water, silicone-in-water, water-in-silicone, oil-in-water-in-oil, oil-in-water-in-silicone emulsions), (both aqueous and hydro-alcoholic); anhydrous bases (such powders); pills; tablets; capsules; and/or the like. Variations and other appropriate vehicles will be apparent to the skilled artisan and are appropriate for use in the present invention.

### A. Oral Formulations

Formulations of the invention suitable for oral administration may be in the form of capsules, cachets, pills, tablets, lozenges, powders, granules, or as a solution or a suspension in an aqueous or non-aqueous liquid, or as an oil-in-water or water-in-oil liquid emulsion, or as an elixir or syrup, and/or the like.

### B. Dietary Supplements, Food Products, Food Additives

Exemplary dietary supplements include one or more of a dietary ingredient such as a vitamin, a mineral, an herb or other botanical, an amino acid, or any other substance used to supplement the diet by increasing total dietary intake, or a concentrate, metabolite, constituent, extract, or any combination thereof. In certain embodiments, dietary supplements are a special category of food and are not a drug. In such embodiments, the composition is included in a consumable carrier. A consumable carrier refers to any carrier or form that can be consumed by a human or animal (e.g., a snack bar, a cookie, a brownie, a muffin, a cracker, a biscuit, a cream or paste, an ice cream bar, a frozen yogurt bar, and/or the like).

Exemplary food products include, but are not limited to, a snack bar, a cookie, a brownie, a muffin, a cracker, a biscuit, a cream or paste, an ice cream bar, a frozen yogurt bar, and/or the like. Exemplary food additives include, but are not limited to, a powder or liquid that can be added to a food product.

The formulation (such as the dietary supplement, a food product, a food additive) can be least 50%, 55%, 60%, 65%, 70%, 75%, 80%, 85%, 90%, 95%, 97.5%, 99.9% natural and/or plant-based. In some embodiments, the formulation can be 100% natural and/or plant-based. Such forms can include a dietary fiber as will be described in further detail below.

### V. Additional Ingredients

The compositions of the present invention of the present invention can be formulated into any suitable composition form that includes or does not include additional ingredients for administration to a human or non-human animal.

The composition can include the myricetin (or a derivative or salt thereof) and/or an anthocyanin (or a derivative or salt thereof) alone or can include the myricetin (or a derivative or salt thereof) and an anthocyanin (or a derivative or salt thereof) and any suitable additional component, such as one or more acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the subject.

### A. Nutraceutical Ingredients

The formulations of the invention can further include various ingredients to help stabilize, or help promote the bioavailability of the components of the beneficial compositions of the invention or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K, folic acid, and/or the like. Non-limiting examples of minerals include iron, calcium, magnesium, potassium, copper, chromium, zinc, molybdenum, iodine, boron, selenium, manganese, and/or the like, and/or derivatives thereof and/or combinations thereof. These vitamins and minerals can be from any source or combination of sources, without limitation. Non-limiting exemplary B vitamins include, without limitation, thiamine, niacinamide, pyridoxine, riboflavin, cyanocobalamin, biotin, pantothenic acid and/or the like, and/or combinations thereof.

Various additives can be incorporated into the present compositions. Optional additives of the present composition include, without limitation, hyaluronic acid, phospholipids, starches, sugars, fats, antioxidants, amino acids, proteins, flavorings, coloring agents, hydrolyzed starch(es) and/or the like, and/or derivatives thereof, and/or combinations thereof.

### B. Pharmaceutical Ingredients

The composition can include pharmaceutical acceptable agents. Non-limiting examples of pharmaceutical active agents include, but are not limited to analgesics, anesthetics, antihistamines, anti-inflammatory agents including non-steroidal anti-inflammatory drugs, antibiotics, antifungals, antivirals, antimicrobials, anti-cancer actives, antineoplastics, biologically active proteins and peptides, enzymes, hemostatics, steroids including hormones and corticosteroids, and/or the like, and/or combinations thereof.

### C. Stabilizing Excipients

In certain preferred embodiments, the formulations described herein can be further stabilized to ensure the stability of the active ingredient incorporated therein. In some embodiments, the stabilizing excipient is selected from sugars, starches, sugar alcohols, and/or the like, and/or mixtures thereof. Examples of suitable sugars for stabilizing excipients include, but are not limited to, trehalose, glucose, sucrose, fructose, and/or the like. Examples of suitable starches for stabilizing excipients include, but are not limited to, hydroxyethyl starch (HES), and/or the like. Examples of suitable sugar alcohols for stabilizing excipients include, but are not limited to, mannitol, sorbitol, and/or the like.

In some embodiments, the stabilizing excipient is present in the formulation in an amount ranging from about 1% (w/v) to about 60% (w/v), from about 1% (w/v) to about 50% (w/v), from about 1% (w/v) to about 40% (w/v), from about 1% (w/v) to about 30% (w/v), from about 1% (w/v) to about 20% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 10% (w/v) to about 60% (w/v), from about 10% (w/v) to about 50% (w/v), from about 10% (w/v) to about 40% (w/v), from about 10% (w/v) to about 30% (w/v), or from about 10% (w/v) to about 20% (w/v). In some embodiments, the stabilizing excipient is present in the formulation in an amount that is about 1%, about 5%, about 10%, about 15%, about 20%, about 25%, about 30%, about 35%, about 40%, about 45%, about 50%, about 55%, or about 60% (w/v).

In some embodiments, the formulation further includes additional stabilizing agents including, for example, antioxidants, chelators, preservatives, and/or the like. Examples of suitable antioxidants include, but are not limited to, ascorbic acid, cysteine, methionine, monothioglycerol, sodium thiosulphate, sulfites, BHT, BHA, ascorbyl palmitate, propyl gallate, N-acetyl-L-cysteine (NAC), Vitamin E. and/or the like. Examples of suitable chelators include, but are not limited to, EDTA, tartaric acid and salts thereof, glycerin, and citric acid and salts thereof. Examples of suitable preservatives include, but are not limited to, benzyl alcohols, methyl parabens, propyl parabens, and/or the like, and/or mixtures thereof. In some embodiments, the formulation further comprises a stabilizing polyol and/or the like.

### D. Excipients

Excipients employed in the compositions of the present invention can be solids, semi-solids, liquids or combinations thereof. The excipients can be solids. Compositions of the invention containing excipients can be prepared by any known technique that includes, for example, admixing an excipient with the myricetin (or a derivative or salt thereof) or the anthocyanin (or a derivative or salt thereof) or a combination of thereof. A composition of the invention contains a desired amount of the myricetin (or a derivative or salt thereof) or the anthocyanin (or a derivative or salt thereof) or a combination of thereof per dose unit and, if intended for oral administration, can be in the form, for example, of a tablet, a caplet, a pill, a hard or soft capsule, a lozenge, a cachet, a dispensable powder, granules, a suspension, an elixir, a dispersion, or any other form reasonably adapted for such administration. Presently preferred are oral dosage forms that are discrete dose units each containing a predetermined amount of the myricetin (or a derivative or salt thereof) or the anthocyanin (or a derivative or salt thereof) or a combination of thereof such as tablets or capsules.

### E. Carriers/Diluents

Suitable carriers or diluents illustratively include, but are not limited to, either individually or in combination, lactose, including anhydrous lactose and lactose monohydrate; starches, including directly compressible starch and hydrolyzed starches (e.g., Celutab™ and Emdex™), mannitol, sorbitol, xylitol, dextrose (e.g., Cerelose™ 2000) and dextrose monohydrate, dibasic calcium phosphate dihydrate, sucrose-based diluents, confectioner's sugar, monobasic calcium sulfate monohydrate, calcium sulfate dihydrate, granular calcium lactate trihydrate, dextrates, inositol, hydrolyzed cereal solids, amylose, celluloses including microcrystalline cellulose, food grade sources of alpha- and amorphous cellulose (e.g., RexcelJ), powdered cellulose, hydroxypropylcellulose (HPC) and hydroxypropylmethylcellulose (HPMC), calcium carbonate, glycine, clay, bentonite, block co-polymers, polyvinylpyrrolidone, and the like. Such carriers or diluents, if present, constitute in total about 5% to about 99.999%, about 10% to about 85%, and 20% to about 80%, of the total weight of the composition. The carrier, carriers, diluent, or diluents selected preferably exhibit suitable flow properties and, where tablets are desired, compressibility.

### F. Disintegrant

Compositions of the invention optionally can include one or more acceptable disintegrants as excipients, particularly for tablet formulations. Suitable disintegrants include, but are not limited to, either individually or in combination, starches, including sodium starch glycolate and pregelatinized corn starches, clays, celluloses such as purified cellulose, microcrystalline cellulose, methylcellulose, carboxymethylcellulose and sodium carboxymethylcellulose, croscarmellose sodium, alginates, crospovidone, and gums such as agar, guar, locust bean, karaya, pectin and tragacanth gums. Disintegrants can be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

### G. Binders

The compositions of the present invention can include binding agents or adhesives particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Such binding agents may also prevent or inhibit crystallization or recrystallization of a co-crystal of the present invention once the salt has been dissolved in a solution. Suitable binding agents and adhesives include, but are not limited to, either individually or in combination, acacia; tragacanth, sucrose, gelatin, glucose, starches such as, but not limited to, pregelatinized starches, celluloses such as, but not limited to, methylcellulose and carmellose sodium, alginic acid and salts of alginic acid; magnesium aluminum silicate, PEG, guar gum, polysaccharide acids, bentonites, povidone, polymethacrylates, HPMC, hydroxypropylcellulose, and ethylcellulose. Such binding agents and/or adhesives, if present, constitute in total about 0.5% to about 25%, preferably about 0.75% to about 15%, and more preferably about 1% to about 10%, of the total weight of the composition. Many of the binding agents are polymers comprising amide, ester, ether, alcohol or ketone groups and, as such, can be included in compositions of the present invention. Polyvinylpyrrolidones is an non-limiting example of a binder used for slow release tablets. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units' ratios in a given polymer affects properties and performance.

### H. Wetting Agents

Wetting agents can be used in the compositions of the present invention. Wetting agent can be selected to maintain the crystal in close association with water, a condition that is believed to improve bioavailability of the composition. Such wetting agents can also be useful in solubilizing or increasing the solubility of crystals. Surfactants can be used as wetting agents. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, poloxamers (polyoxyethylene and polyoxypropylene block copolymers), polyoxyethylene fatty acid glycerides and oils, for example polyoxyethylene (8) caprylic/capric mono- and diglycerides, polyoxyethylene (35) castor oil and polyoxyethylene (40) hydrogenated castor oil, polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80, propylene glycol fatty acid esters, for example propylene glycol laurate, sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

### I. Lubricants

Lubricants can be included in the compositions of the present invention. Suitable lubricants include, but are not limited to, either individually or in combination, glyceryl behapate, stearic acid and salts thereof, including magnesium, calcium and sodium stearates; hydrogenated vegetable oils, colloidal silica, talc, waxes, boric acid, sodium benzoate, sodium acetate, sodium fumarate, sodium chloride, DL-leucine, PEG (e.g., Carbowax™ 4000 and Carbowax™ 6000 of the Dow Chemical Company), sodium oleate, sodium lauryl sulfate, and magnesium lauryl sulfate. Such lubricants, if present, constitute in total about 0.1% to about 10%, preferably about 0.2% to about 8%, and more preferably about 0.25% to about 5%, of the total weight of the composition.

### J. Other Ingredients

Surfactant, emulsifier, or effervescent agents can be used in the compositions. Emulsifying agents can be used to help solubilize the ingredients within a soft gelatin capsule. Non-limiting examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

The formulations of the invention can further include various ingredients to help stabilize, or help promote the bioavailability of the components of the beneficial compositions of the invention or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals can be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Various additives can be incorporated into the present compositions. Optional additives of the present composition include, without limitation, hyaluronic acid, phospholipids, starches, sugars, fats, antioxidants, amino acids, proteins, flavorings, coloring agents, hydrolyzed starch(es) and derivatives thereof or combinations thereof.

The formulation can include a dietary fiber. Examples of dietary fibers for use in the invention include, but are not limited to, the dietary fiber is indigestible dextrin, polydextrose, soybean-derived water-soluble dietary fiber, hydrolyzed guar gum, glucomannan, inulin, pectin, or sodium alginate, and/or the like, and/or any combination thereof. In some embodiments, the dietary fiber contributes to the bioavailability of the composition.

### VI. Pharmaceutical Compositions

The active agents of the present invention (e.g., myricetin or a derivative or salt thereof or an anthocyanin or a derivative or salt thereof) can be formulated as pharmaceutical compositions ready for storage or administration, typically comprising a therapeutically effective amount of an active agent used in the context of the invention, in a pharmaceutically acceptable carrier. In some embodiments, the pharmaceutical composition is formulated to cause slow release of the active agents.

The therapeutically effective amount of a compound of the present invention will depend, for example, the type of mammal being treated, and the physical characteristics of the specific mammal under study. These factors and their relationship to determining this amount are well known to those skilled in the art. This amount and method of administration can be tailored for optimal efficacy, and may depend on factors such as weight, diet, concurrent medication, and other factors well known to those skilled in the medical field. The most appropriate dose sizes and dosage regimen for use in humans can be guided by the results obtained in the present invention and can be confirmed in appropriately designed clinical trials.

Through conventional means, an effective dosage and treatment protocol can be determined, starting with a low dose in laboratory animals and then increasing the dosage while monitoring for effects, as well as systematically modifying the dosage regimen. In determining an optimal dosage for a given subject, the physician may take into account numerous factors. The expert knows how to take it into account. The term “pharmaceutically acceptable carrier” includes any of the standard pharmaceutical carriers. Pharmaceutically acceptable carriers for therapeutic use are well known in the pharmaceutical art and are described, for example, in Remington's Pharmaceutical Sciences, Mack Publishing Co. (A. R. Gennaro ed. 1985). For example, sterile saline and phosphate buffered saline at slightly acidic or physiological pH can be used. Suitable pH buffering agents may be, for example, phosphate, citrate, acetate, lactate, maleate, tris-(hydroxymethyl)-aminomethane (TRIS), W-Tris(hydroxymethyl)methyl-3-aminopropanesulfonic acid (TAPS), bicarbonate ammonium, diethanolamine, histidine, which in certain embodiments is a preferred buffer, arginine, lysine, or acetate, or mixtures thereof. The term further encompasses any agent listed in the United States Pharmacopeia for use in animals, including humans.

The term “pharmaceutically acceptable salt” refers to a salt of the compound. Salts include pharmaceutically acceptable salts, such as, for example, acid addition salts and base salts. Examples of acid addition salts include hydrochloride salts, citrate salts, and acetate salts. Examples of basic salts include salts where the cation is selected from alkali metals such as sodium and potassium, alkaline earth metals such as calcium, and ammonium ions N (R3) 3 (R4), where R3 and R4 independently denote C 1 optionally substituted-6, optionally substituted C 2-6 alkenyl, optionally substituted aryl, or optionally substituted heteroaryl. Other examples of pharmaceutically acceptable salts are described in “Remington's Pharmaceutical Sciences”, 17th edition. Ed. Alfonso R. Gennaro (Ed.), Mark Publishing Company, Easton, PA, USA, 1985 and newer editions, and in the Encyclopedia of Pharmaceutical Technology.

Pharmaceutical compositions of the invention may be in unit dosage form. In such form, the preparation is subdivided into unit doses containing appropriate quantities of the active component. The unit dosage form may be a packaged preparation, the package containing different quantities of the preparations, for example, tablets, capsules and powders packaged in vials or ampoules. The unit dosage form can also be a capsule, cachet, or tablet itself, or it can be the appropriate number of any of these package forms. The compositions may be formulated by any suitable route and means of administration. Pharmaceutically acceptable carriers or diluents include those used in formulations suitable for oral administration. The formulations may conveniently be presented in unit dosage form and may be prepared by any of the methods well known in the art of pharmacy.

### VII. Enhanced Bioavailability

The composition can be included in a formulation that increases the bioavailability of the active ingredients of the composition to the subject. Examples of such formulations include encapsulation of the composition, for example, microencapsulation and nanoencapsulation, by methods known in the art. Microencapsulation or nanoencapsulation into particles can improve bioavailability profiles of the active agents and/or prevent degradation in gastric fluid. Microencapsulation or nanoencapsulation may be by liposomal encapsulation, such that the active agents are present inside particles having lipid walls. Other encapsulation methods may be used.

Other methods to enhance bioavailability can include, but not be limited to, lipid-based formulation approaches, particularly the self emulsifying drug delivery system (SEDDS) or selfmicroemulsifying drug delivery system (SMEDDS), or selfnanoemulsifying drug delivery systems (SNEDDS). The formulations described herein contain a therapeutic agent in substantially solubilized form. The formulations improve the bioavailability and pharmacokinetic profile of the therapeutic agent after oral administration. SEDDS/SMEDDS/SNEDDS formulations are isotropic mixtures of an oil, a surfactant, a cosurfactant (or solubilizer), and a drug. The basic principle of this system is its ability to form fine oil in-water (o/w) microemulsions under gentle agitation following dilution by aqueous phases (i.e., the digestive motility of the stomach and intestine provide the agitation required for selfemulsification in vivo in the lumen of the gut). This spontaneous formation of an emulsion in the gastrointestinal tract presents the drug in a solubilized form, and the small size of the formed droplet provides a large interfacial surface area for drug absorption. Apart from solubilization, the presence of lipid in the formulation further helps improve bioavailability by affecting the active agent absorption. Selection of a suitable self-emulsifying formulation depends upon the assessment of (1) the solubility of the active agent in various components, (2) the area of the self-emulsifying region as obtained in the phase diagram, (3) the droplet size distribution of the resultant emulsion following self-emulsification, and (4) the release rate of the active after dispersion in intestinal fluids.

It is also agreed that the amounts of the active ingredients can be reduced when methods to increase bioavailability are used. For example, amounts can be reduced by about 10%, 50%, 100%, 150%, 200%, 250%, 300%, 350%, 400%, 450%, 500%, 750%, 1000% relative to amounts already stated throughout this specification.

### A. Modified Release

The invention provides an oral composition in a modified release formulation having release profiles to release the substance in an active form at one or more sites along the gastrointestinal (GI) tract for modified release. The invention provides an oral pharmaceutical composition comprising formulations comprising active agents such as myricetin (or a derivative or salt thereof) or an anthocyanin (or a derivative or salt thereof) or a combination of thereof in, for example, a liquid, semi-solid or solid core capsule format, the capsule having release profiles to release active agents at one or more sites along the gastrointestinal tract where absorption is maximized or therapeutic efficacy is maximized.

The modified release formulation may have one layer and may be solid throughout. Alternatively, the formulation may have two layers comprising a solid outer shell layer encapsulating a liquid, semi-solid or solid core. For example, the formulation may have three layers comprising a solid outer shell layer; a solid, semi-solid or liquid middle buffer layer; and a liquid, semi-solid or liquid core.

The formulations may be modified to enable modified release of the active agent(s). For example, a modified release coating may be applied to the outer shell layer of the formulation. Alternatively, an outer shell layer of the formulation may be modified to achieve modified release. In other formats, the formulation core or entirety may control the rate of active agents. For example a buffer layer of the formulation may be modified to achieve modified release. Alternatively, the liquid, semi-liquid or solid core of the formulation may be modified to achieve modified release. For example, polymeric materials may be used achieve modified release such as polymeric materials that are sensitive to one or more of pH, time, thickness, erosion, and bacterial breakdown.

The active agent(s) (e.g., myricetin or a derivative or salt thereof or an anthocyanin or a derivative or salt thereof) can be in micronized or nanosized particles. The active agent(s) may be in soluble form. Alternatively, the active agent(s) may be in crystalline form or the active agent(s) may be in amorphous form.

### VIII. Methods of Use

Aspects of the invention relate to methods related to using the compositions described here in a variety of applications to a subject such as weight management, treatment/prevention of a metabolic disorder, and/or treatment/prevention of addiction. The methods can include administering the composition, formulation, pharmaceutical composition, dietary supplement, food product, and/or food additive disclosed herein to the subject.

In some embodiments, the methods described herein further include the step of identifying a patient (e.g., a subject) in need of such treatment (e.g., by way of blood assay, body mass index, genetic testing, or other conventional method known in the art). In some embodiments, the methods described herein further include the step of identifying a patient that has a disease, disorder, or condition as provided here (e.g., a GLP-1 associated disease, disorder, or condition).

### A. Weight Management

Methods for weight management are provided herein. may be used in a method for treating or preventing weight gain or obesity, promoting weight loss, appetite suppression, modifying satiety, modifying fat uptake, increasing metabolism to promote weight loss or prevent weight gain, maintaining body weight, reducing body fat or fatty tissues, increasing muscle or lean body mass, reducing hepatosteatosis, improving fatty liver, improving one or more liver NASH scores, enhancing fatty acid metabolism in liver, promoting a healthy lipid profile (by, e.g., lowering LDL cholesterol, lowering total cholesterol, lowering triglyceride, or increasing HDL), promoting glucose metabolism, reducing fasting glucose levels, maintaining healthy glucose levels, reducing caloric intake, improving caloric efficiency, reducing food intake, reducing visceral fat, reducing waist circumference, reducing body-to-mass index (BMI), increasing energy, increasing stamina, maintaining energy level while dieting, promoting thermogenesis reducing excess fluids, reducing water retention while maintaining normal hydration, increasing muscle mass, improving fat-to-muscle mass ratio, optimizing or improving body composition, optimizing or improving hormonal balance for appitite control, maintaining normal insulin, leptin, ghrelin, PYY, GIP or enterostatin levels or functions, optimizing, managing or improving hormonal balance to control satiety, maintaining or managing healthy CCK peptide, GLP-1, bombesin, or somatostatin levels or functions, maintaining healthy flora of intestinal tract, optimizing, improving or managing digestion, inducing lipolysis, reducing intracellular triglyceride accumulation, reducing fat accumulation in adipose tissue or an adipocyte, maintaining healthy adiponectin levels, managing or reducing lipogenesis or weight gain associated with metabolism of fructose, glucose or both, reducing or controlling oxidative stress associated with an overweight or obese mammal (e.g., by reducing reactive oxygen species or oxidative free radicals; improving ORAC (Oxygen Radical Absorption Capacity) values; maintaining a healthy level of glutathione, superoxide dismutase, catalase, peroxidase or endogenous antioxidants; maintaining healthy oxidative homeostasis), controlling or managing systemic inflammation associated with an overweight or obese mammal (e.g., by promoting normal metabolism of arachidonic acid, maintaining a normal level of pro-inflammatory cytokines), managing mood stress or other mental disorders associated with an overweight or obese mammal, and/or any combination thereof.

In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates weight management. In some embodiments, a reduction in BMI to about or below 40, about or below 30, or about or below 20 indicates weight management.

In some embodiments, the weight management methods are used in a subject diagnosed or suspected to have a metabolic disorder as described further below.

### B. Treatment of a Metabolic Disorder

Also provided are methods of prevention and or/treatment of a metabolic disorder in a subject. The metabolic disorder can be diabetes or a diabetes related disorder, or obesity or an obesity related disorder. The link between obesity and diabetes is well known, so these conditions are not necessarily separate or mutually exclusive.

Diabetes related disorders include insulin resistance, glucose intolerance, increased fasting 10 glucose, pre-diabetes, type 1 diabetes, type 2 diabetes, gestational diabetes hypertension, dyslipidemia, bone related disorders, and/or the like, and/or combinations thereof.

Diabetes related disorders also include atherosclerosis, arteriosclerosis, coronary heart disease, peripheral artery disease and stroke; or conditions associated with atherogenic dyslipidemia, blood fat disorders, elevated blood pressure, hypertension, a prothrombotic state, a proinflammatory state, and/or the like, and/or combinations thereof.

### (1) Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, insulinoma, hypothyroidism, obese type II diabetes, pseudohypoparathyroidism, hypogonadism), hypothalamic obesity, hereditary obesity (e.g., Prader-Willi syndrome, Laurence-Moon-Biedl syndrome), and/or drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, urine incontinence, and/or metabolic syndrome. In some embodiments, the compositions described herein can be used to treat patients exhibiting symptoms of both obesity and insulin deficiency.

### (2) Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

In some embodiments, the compositions and methods for treating a patient with a condition, disease, or disorder (such as diabetes) described herein reduce fasting plasma glucose levels, reduce non-fasting plasma glucose levels, reduce HbA1c levels, reduce glucagon levels, increase insulin levels, and/or reduce BMI.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, cognitive dysfunction, and systemic inflammation.

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity).

### (3) Disorders of Metabolically Important Tissues

In some embodiments, the metabolic disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, hyperlipoproteinemia, glycogen storage diseases, Weber-Christian disease, Wolman disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and can ultimately lead to cirrhosis (Skelly et al., J Hepatol 2001; 35:195-9; Chitturi et al., Hepatology 2002; 35 (2): 373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl: S186-90).

Other non-limiting examples of disorders in metabolically important tissues include joint disorders (e.g., osteoarthritis, secondary osteoarthritis), steatosis (e.g., in the liver); fibrosis (e.g., in the liver); cirrhosis (e.g., in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging, pregnancy, protection against bone fractures, and malnutritionpolycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, any of the compositions or formulations described herein (e.g., those including myricetin or a derivative or salt thereof and an anthocyanin or a derivative or salt thereof) can be used for treating surgical trauma by improving recovery after surgery and/or by preventing the catabolic reaction caused by surgical trauma.

### C. Addiction

Methods for treatment of addition and alcoholism are provided herein. The disclosure provides a method of treating a substance use disorder in a subject, comprising administering to the subject any or the compositions or formulations described herein disclosed herein (e.g., those including myricetin or a derivative or salt thereof and an anthocyanin or a derivative or salt thereof).

In some embodiments of the methods of the disclosure, the substance use disorder can include abuse of alcohol, marijuana, synthetic cannabinoids, opioids, stimulants, barbiturates, benzodiazepines, dextromethorphan (DXM), a sleep medication, khat, synthetic cathinones, cocaine, 3,4-methylenedioxymethamphetamine (MIDMA), phencyclidine (PCP), lysergic acid diethylamide (LSD), psilocybin, an inhalant, Rohypnol, gamma-hydroxybutyric acid (GHB), N,N-Dimethyltryptamine (DMT), ayahuasca, mescaline, salvia, or nicotine. In some embodiments of the methods of the disclosure, the substance use disorder comprises abuse of opioids. In some embodiments of the methods of the disclosure, the substance use disorder includes abuse of alcohol/alcoholism.

In some embodiments of the methods of the disclosure, the opioid comprises Heroin, Codeine, Fentanyl, Hydrocodone (Dihydrocodeinone), Hydromorphone, Meperidine, Methadone, Morphine, Oxycodone or Oxymorphone. In some embodiments, the stimulant comprises Amphetamine, Amphetamine sulfate, Methamphetamine, Dextroamphetamine, Levoamphetamine, Lisdexamfetamine, Atomoxetine, Methylphenidate, Dexmethylphenidate, Oxymetazoline, Pseudoephedrine, Phenylephrine, or the like, and/or a combination thereof. In some embodiments, the benzodiazepine comprises Aprazolam, Chlorodiazepoxide, Diazepam, Lorazepam, Triazolam, or the like, and/or any combination thereof. In some embodiments, the barbiturate comprises Phenobarbital, Pentobarbital, Methohexital, Secobarbital, Butabarbital, Butalbital, or the like, and/or any combination thereof. In some embodiments, the sleep medication comprises Eszopiclone, Zaleplon, Zolpidem, or the like, and/or any combination thereof.

In some embodiments of the methods of the disclosure, administering any or the compositions or formulations described herein disclosed herein (e.g., those including myricetin or a derivative or salt thereof and an anthocyanin or a derivative or salt thereof) reduces a symptom of withdrawal or prevents a relapse of the substance use disorder in the subject over a period of time, for example over 0.5, 1, 2, 3, 4, 5, years or more.

### D. Subjects

In some embodiments, the subject of the method is a subject determined to have, or suspected to have any of the diseases, disorders, conditions (e.g., metabolic condition or addiction) or clinical characteristics (e.g., weight, BMI, vital signs, genetic testing, etc.) disclosed herein.

In some embodiments, the subject is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

### E. Dosages and Protocols

As one of ordinary skill in the art would recognize, effective dosages can vary based on a number of parameters and particular dosages can be determined by methodologies known in the art. Generally, therapeutically effective amounts of the disclosed compositions or active ingredients therein vary from about 0.001 mg/kg body weight to about 10 g/kg body weight (e.g., about 0.005, 0.01, 0.05, 0.1, 1.0, 10.0, 100, 250, or 500 mg/kg body weight). According to some such embodiments, the therapeutically effective amount of a composition comprising myricetin (or a derivative or salt thereof) or an anthocyanin (or a derivative or salt thereof) or a combination of thereof is at least or about 0.5 mg/kg per body weight of myricetin (or a derivative or salt thereof) (e.g., at least or about 0.5, 1, 5, 10, 15, 20, 25, 30, 35, 40, 50, 55, 60, 65, 70, 75 or more mg/kg body weight) and/or at least or about 0.25 mg/kg per body weight anthocyanin (or a derivative or salt thereof) (e.g., at least or about 0.5, 1, 5, 10, 15, 20, 25, 30, 35, 40, 50, 55, 60, 65, 70, 75 or more mg/kg body weight). These dosages are pertinent regardless of the precise content or intended use.

In some embodiments, the dosage is administered at any interval to achieve a desired effect, such as, but not limited to, 3 times/day, 2 times/day, daily, 6/week, 5/week, 4/week, 3/week, 2/week, 1/week, or more or less.

An effective dosage and treatment protocol can be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person. The term pharmaceutically acceptable carrier includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate buffered saline at slightly acidic or physiological pH may be used. Suitable pH buffering agents may be, e.g., phosphate, citrate, acetate, lactate, maleate, tris/hydroxymethyl)aminomethane (TRIS), Λ/-Tris(hydroxymethyl)methyl-3aminopropanesulphonic acid (TAPS), ammonium bicarbonate, diethanolamine, histidine, which in certain embodiments is a preferred buffer, arginine, lysine, or acetate or mixtures thereof. The term further encompasses any agents listed in the US Pharmacopeia for use in animals, including humans.

### EXAMPLES

The following examples are offered to illustrate, but not to limit, the claimed invention.

### I. Example 1

Experiments will be done to compare the effects of (i) a composition of myricetin and an anthocyanin, (ii) a composition of an anthocyanin, (iii) a composition of myricetin, and (iv) no treatment on metabolic parameters in an animal model of obesity. Animals will be administered the composition and weight, lean muscle mass, insulin resistance, metabolic rate, brown adipose tissue, plasma lipids will be measured over a period of time. Synergistic effects of myricetin and an anthocyanin will be observed.

### II. Example 2

Experiments will be done to compare effects of various dosages of the disclosed composition. A ratio of myricetin to an anthocyanin between 3:1 and 7:1 will be found to be most effective.

### III. Example 3 (Prophetic Example)

Experiments will be done related to (1) dose analysis validation and stability; (2) toxicology studies; and (3) metabolism studies of a composition of myricetin and an anthocyanin in vivo. Non-GLP dose range finding (DRF) studies are conducted, followed by pivotal repeat dose studies of 13-week duration. Dose administration will be twice per day. Included will be treatment-free of 2 weeks in the control and high dose groups period to assess recovery from any treatment-related findings. The experiments will be conducted in rats and/or dogs. Administration will be via the oral (gavage) route of administration as this route will be used for clinical administration. HPLC methodology will be used for providing analytical support for confirming the stability, homogeneity and achieved concentration of formulations for use in nonclinical studies.

LC-MS/MS methodology will be used to assess plasma levels in PK/TK blood samples collected from the in vivo studies. A method will be developed and then used to analyze the samples collected from non-GLP PK and/or DRF studies. This allows for the review of the plasma concentration-time data obtained prior to validation. If needed, the analytical range will be adjusted to ensure that the validated analytical range encompasses the concentrations expected in the GLP toxicology studies.

### IV. Example 4 (Prophetic Example)

A study for demonstrating the safety profiles of a composition of myricetin and an anthocyanin will be done. The studies use an in vitro human hepatocyte system and in vivo rat models. The data obtained in the study will be used to support other available information on safety of the composition for potential GRAS or NDI notification.

Study design. Aim 1. An in vitro study utilizing human HepaRG cells and Aim 2. An in vivo study including two stages and will consider use of a rat model (F344/N, Wistar or Sprague-Dawley strain).

Aim 1. An in vitro safety study utilizing human HepaRG cells culture will be conducted. HepaRG cells will be treated with five doses of the composition for 96 h. After treatment, the media will be collected and flash-frozen and stored at −80° C. for biochemical analyses on markers of hepatocyte injury (namely: lactate dehydrogenase, LDH; and alanine and aspartame aminotransferases, ALT and AST, respectively). The cells will be harvested, flash-frozen and stored at −80° C. for subsequent molecular (transcriptomic) analysis.

Aim 2. An in vivo safety study using the rat model of F344/N, Wistar or Sprague-Dawley strain will be conducted. Rat will be a preferred species for toxicological studies submitted for consideration by the U.S. FDA and the National Toxicology Program and three abovementioned strains are classical strains that will be used for this purpose. Aim 2 will be performed in two stages:

Stage 1: Acute. The product will be administered with a single dose to identify the marginal dose and doses suitable for sub-chronic studies. Groups of three female rats/group will be administered with 0, 300 and 2,000, mg/kg via oral gavage (9 rats). Animals will be inspected at 1, 2, 4, and 24 h and then daily for the duration of 14 days for any signs of toxicity.

Stage 2: Sub-chronic (or, so called “90-day study”). The rats will receive powdered product pre-mixed in their diet for 13 consecutive weeks. Groups of ten male and ten female rats will receive 0, 300, 1,000, or 2,000 mg/kg with food (40 male, 40 female rats). These doses may be corrected based on the results obtained in Stage 1. Animals will be monitored daily (5 days/week), with body weights, food and water consumption recorded weekly. Animals will be terminated 90 days after the initiation of the study. A separate group of 5 male and 5 female rats in the groups receiving 0, or 2,000 mg/kg (10 male, 10 female rats) receive normal chow for the duration of 28 days to address reversibility of any potential alterations.

Stages 1 and 2 incorporate two studies recommended for testing of any product intended for ingestion by humans by the Organization for Economic Cooperation and Development (OECD).

End-points. At necropsy, body weights will be recorded and major internal organs (liver, heart, kidneys, lungs, and intestines) will receive gross exanimation for visual evidence of toxicity (i.e. increase in size) and visual lesions. Any abnormally looking organs/tissues will be fixed for further histopathological evaluation. Liver, heart and kidneys tissue will be assessed in detail. After that, these three tissues will be: 1) fixed for further histopathological examination, 2) flash-frozen in liquid nitrogen for further molecular and biochemical investigations, if needed. Additionally, blood and urine specimens will be collected for blood biochemistry profiling and urinalysis as required by the OECD.

### V. Example 5 (Prophetic Example)

A randomized, double-blind, placebo-controlled, parallel study to evaluate the safety, tolerability, and effects of a composition of myricetin and an anthocyanin on muscle mass and function in adults will be conducted. Two populations aged ≥18 and ≤60 years participate; Type II diabetic group and pre-diabetic adult group (50/50) participate.

Number of participants: Telephone screen: n=492; Screened: n=246 (estimate); Randomised: n=176:4 Arms with 44 participants per group; Evaluable: approx. n=140 participants (estimated 20% dropout/withdrawal based on study duration).

Objective 1: To assess the safety and tolerability of 12-week daily administration of dietary supplement as assessed by occurrence of any adverse events (AEs).

### Endpoint Variable 1: Incidence of AEs by Product Causality During 12-Weeks of Study Product Intake.

Objective 2: To determine the safety, tolerability and effects of 12-week daily administration of dietary supplement on safety blood profile, vitals, adverse events, body weight, fat mass, HbA1c, blood glucose, and inflammation marker: hs-CRP.

### Endpoint Variable 2:

Change in Safety blood profile (Serum Chemistry Profile and Haematology Profile—Full blood count) from baseline (Week 0) to the end of the intervention (Week 12) in the pre-diabetic and diabetic group, compared to Placebo.
Change in systolic blood pressure (mmHg), diastolic blood pressure (mmHg), heart rate (BPM) and body temperature (° C.) parameters from baseline (Week 0) to the end of the intervention (Week 12) in the pre-diabetic and diabetic group, compared to the Placebo.
Incidence of SAEs per dose by product causality
Incidence of product interruption by product causality.
Incidence of product discontinuation by product causality
Change in body weight (kg) from baseline (Week 0) to the end of the intervention (Week 12) in the pre-diabetic and diabetic group, compared to the Placebo.
Change in Total Body Fat mass, Total Lean Mass and total mass as measured by DEXA body composition analysis from baseline (Week 0) to the end of the intervention (Week 12) in the pre-diabetic and diabetic group, compared to the Placebo.
Change in plasma HbA1c (mmol/mol) from baseline (Week 0) to the end of the intervention (Week 12) in the pre-diabetic and diabetic group, compared to the Placebo.
Change in blood glucose (mmol/L) from baseline (Week 0) to the end of the intervention (Week 12) in the pre-diabetic and diabetic group, compared to the Placebo.
Change in hs-CRP from baseline (Week 0) to the end of the intervention (Week 12) in the pre-diabetic and diabetic group, compared to the Placebo.
50% of the diabetic group are subjected to continuous glucose monitoring (CGM) for the duration of the study. The CGM device is worn during the 2 weeks run in period for baseline and prior to 2 weeks to visit 3 for the end of intervention.

Participants: To be eligible for inclusion, the participant must fulfil all of the following criteria: be able to give written informed consent; be aged ≥18 and ≤60 years; BMI≥30.0 kg/m2; Type II diabetic group: HbA1c above 6.5%, with current use of metformin; Pre-diabetic group: HbA1c<5.7%, with no drug treatment; is in general good health, as determined by the investigator; maintain current level of physical activity; willing to consume the study product daily for the duration of the study.

Participants will be excluded from the study if they meet any of the following criteria: participants who are pregnant or wish to become pregnant during the study or who are lactating and/or currently breastfeeding; participants currently of biological childbearing potential but not using a continuous effective method of contraception as determined by the investigator; has a history of drug and/or alcohol abuse; are hypersensitive to any of the components of the Study Product; participants with uncontrolled hypertension, uncontrolled hyperlipidaemia, inflammatory disorders, or any condition which contraindicates, in the investigator's judgement, entry to the study; has any significant acute or chronic coexisting health conditions that would prevent them from fulfilling the study requirements, put the Participant at risk or would confound the interpretation of the study results will be judged by the investigator on the basis of medical history and routine laboratory test results.

Excluded health conditions include: Type 1 Diabetes; Immunocompromised health condition; Currently or recently taking a medication that the investigator believes would interfere with the objectives of the study or pose a safety risk or confound the interpretation of the study results.

Prohibited medications include: GLP-1 receptor agonists (in the past 6 months); Current or recent (in the past 4 weeks prior to visit 1) use of prohibited nutritional and non-nutritional supplements, including: Dietary supplements for weight loss; Individuals who, in the opinion of the investigator, are considered to be poor attendees or unlikely for any reason to be able to comply with the study.

Participants may not be participating in other clinical studies. If the participant has previously taken part in an experimental study, the Investigator must ensure sufficient time has elapsed before entry to this study to ensure the integrity of the results.

### VI. DEXA

Dual-Energy X-ray Absorptiometry (DEXA or DXA) is a medical imaging technique primarily used to measure bone mineral density (BMD) and body composition (fat and lean mass). It uses low-dose X-rays at two different energy levels to create detailed images of bones and tissues.

### VII. 3-Day Food Diary

Participants will complete a 3-Day Food Diary, logging any food eaten for three days prior to Visit 2 to establish their dietary habits at Baseline, and again at Visit 3 to reassess it at end of intervention. This data will be used to confirm that Participants will maintain their habitual diet throughout the study via evaluation of key dietary components at baseline compared to end-of-intervention (i.e., energy, total carbs, total sugar, fiber, and fat).

All statistical elements of the study will be informed by the principles of the International Conference on Harmonisation Good Clinical Practice (ICH-GCP) documents, and executed by the study statistician under the standard operating procedures for Atlantia Clinical Trials Ltd. A detailed Statistical Analysis Plan (SAP) will be drafted and shared with sponsor prior to LPLV, to be approved prior to database lock. The SAP will be developed by the study statistician, in close collaboration with the study PI and study sponsor.

Reporting of the statistical results will be informed by the principles of ICH-GCP and CONSORT. Summary Descriptive tables will include sample size, the minimum and All statistical elements of the study will be informed by the principles of the International Conference on Harmonisation Good Clinical Practice (ICH-GCP) documents, and executed by the study statistician under the standard operating procedures for Atlantia Clinical Trials Ltd. A detailed Statistical Analysis Plan (SAP) will be drafted and shared with sponsor prior to LPLV, to be approved prior to database lock. The SAP will be developed by the study statistician, in close collaboration with the study PI and study sponsor.

Reporting of the statistical results is informed by the principles of ICH-GCP and CONSORT. Summary Descriptive tables will include sample size, the minimum and maximum statistics, mean, median, quartiles and standard deviation for each endpoint. Inferential analysis is completed to assess change over time between/within products. Test selection is decided during protocol development.

### VIII. Blood Samples

Safety parameters at screening, baseline and end of study include:
Chemistry: ALT, AST, Blood Urea Nitrogen (BUN), Chloride, Creatinine, Potassium, Total Protein, Sodium
FBC: White Blood Cells (WBC), Red Blood Cells (RBC), Hemoglobin, Hematocrit, Platelets, MCV, MCH, MCHC, RDW
Glucose: week 0 and week 12
hs-CRP: week 0 and week 12
HbA1c: screening, week 0 and week 12

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents and PCT publications are incorporated herein by reference for all purposes.

What is claimed:

1. A GLP-1 agonist composition, the composition comprising therapeutically effective amounts of myricetin or a derivative or salt thereof and an anthocyanin or a derivative or salt thereof, wherein the myricetin or the derivative or salt thereof and the anthocyanin or a derivative or salt thereof is present in the composition at a weight ratio of 8:1 to 3:1.

2. The composition of claim 1, wherein the anthocyanin or the derivative or salt thereof is cyanidin 3-glucoside.

3. The composition of claim 2, wherein the myricetin or the derivative or salt thereof and the anthocyanin or a derivative or salt thereof is present in the composition at a weight ratio of 6:1 to 4:1.

4. The composition of claim 3, wherein the myricetin or the derivative or salt thereof and the anthocyanin or a derivative or salt thereof is present in the composition at a weight ratio of 5:1.

5. The composition of claim 2, wherein the myricetin or the derivative or salt thereof and the anthocyanin or a derivative or salt thereof is present in the composition at a weight ratio of 7.25:1 to 5.25:1.

6. The composition of claim 5, wherein the myricetin or the derivative or salt thereof and the anthocyanin or a derivative or salt thereof is present in the composition at a weight ratio of 6.25.

7. The composition of claim 1, wherein the composition is capable of reducing a person's weight or limiting muscle loss in the person.

8. The composition of claim 1, wherein the composition comprises 100 mg to about 5000 mg myricetin or derivative or salt thereof and about 50 mg to 3000 mg of the anthocyanin or derivative or salt thereof.

9. The composition of claim 1, wherein the composition comprises 1875 or 1500 mg myricetin or the derivative or salt thereof and 300 mg of the anthocyanin or the derivative or salt thereof.

10. The composition of claim 1, wherein the composition is encapsulated.

11. The composition of claim 1, wherein the composition is in oral dosage form.

12. The composition of claim 11, wherein the oral dosage form is a capsule, a tablet, a caplet, a gel caplet (gelcap), a syrup, a liquid, or a powder, or any combination thereof.

13. The composition of claim 1, wherein the composition is for weight management, treatment of a metabolic disorder, and/or treatment of an addiction in a subject.

14. The composition of claim 13, wherein the weight management comprises one or more of weight loss, maintenance of weight loss, preserving lean body mass, minimizing loss of lean body mass during weight loss, decreased food consumption, increasing satiety, reducing pre-meal hunger, reducing intra-meal food intake, or prevention of weight gain in the subject.

15. The composition of claim 1, wherein the myricetin or a derivative or salt thereof or the anthocyanin or a derivative or salt thereof is at least 80% pure.

16. The composition of claim 1, wherein the composition does not include quercetin and/or chlorogenic acid.

17. A method for managing weight, maintaining cardiovascular health, treating a metabolic disorder, or addiction in a subject, the method comprising administering the composition of claim 1 to the subject.
